(19) United States
(12) Patent Application Publication
(10) Pub. No.: US 2026/0166084 A1
(43) Pub. Date: Jun. 18, 2026

(54) NOVEL DRUG-INDUCIBLE DEGRADATION TAGS

(71) Applicants: THE INSTITUTE OF CANCER RESEARCH: ROYAL CANCER HOSPITAL, London (GB); CANCER RESEARCH TECHNOLOGY LIMITED, London (GB); UCL Business Ltd, London (GB)

(72) Inventors: Habib BOUGUENINA, London (GB); Yann-Vai LE BIHAN, London (GB); Louis CHESLER, London (GB); Stephanos NICOLAOU, London (GB); John ANDERSON, London (GB); Ian COLLINS, London (GB)

(21) Appl. No.: 19/127,093

(22) PCT Filed: Nov. 7, 2023
(86) PCT No.: PCT/GB2023/052906

§ 371 Date: May 2, 2025

(30) Foreign Application Priority Data
Nov. 7, 2022 (FR) FR2211540

(51) Int. Cl. A61K 35/17; A61K 31/454; A61K 31/513; A61K 31/5377; A61K 40/11; A61K 40/31; A61K 40/42; A61P 35/00; C07K 14/705; C07K 14/725; C07K 16/28; C12N 15/86

(52) U.S. Cl. CPC A61K 35/17; A61K 31/454; A61K 31/513; A61K 31/5377; A61K 40/11; A61K 40/31; A61K 40/421; A61P 35/00; C07K 14/7051; C07K 14/70517; C07K 16/2827; C12N 15/86; A61K 2239/13; A61K 2239/21; A61K 2239/22; C07K 2317/622; C07K 2319/81; C07K 2319/95; C12N 2740/15043

(57) ABSTRACT

The present invention provides novel drug-inducible degradation tags (degrons), as well as fusion proteins, cells and pharmaceutical compositions comprising the same. Nucleic acid sequences and vectors encoding the novel degrons are also provided. Methods of using the novel degrons, fusion proteins, cells, pharmaceutical compositions, nucleic acid sequences and vectors are also provided herein.

The present invention provides novel drug-inducible degradation tags (degrons), as well as fusion proteins, cells and pharmaceutical compositions comprising the same. Nucleic acid sequences and vectors encoding the novel degrons are also provided. Methods of using the novel degrons, fusion proteins, cells, pharmaceutical compositions, nucleic acid sequences and vectors are also provided herein.

### BACKGROUND

Chemical biology tools that induce targeted protein degradation with drug like precision have gained a lot of traction in recent years due to their ability to modulate protein levels acutely and reversibly in cells. These technologies are based on the use of small amino acid tags fused to a protein of interest that will induce its degradation in the presence of a specific chemical compound. Several technologies are currently available, examples include small-molecule displacement of a cryptic degron¹, Small Molecule-Assisted Shutoff (SMASh)², degradation of a fused hydrophobic tag (HaloTag), the auxin-inducible degron (AID1/2), the HaloPROTAC³ and the dTAG systems^{4,5}. These tools have enabled multiple discoveries at the pre-clinical level but their various limitations (need to express multiple components, poor PK/PD of the associated compounds, use of non-FDA approved molecules) have hindered translation to more clinically relevant applications.

One of the best described examples of drug-induced protein degradation is the mechanism of action of the thalidomide-like derivatives (called immunomodulatory imide drugs IMiDs, or more recently, Cereblon E3 ligase modulators CELMoDs). These degrader drugs bind to a conserved tri-tryptophan cage on the surface of Cereblon (CRBN), the substrate receptor of the Cullin4-RING E3 ubiquitin ligase CRL4^{CRBN}, and induce the alteration of CRL4^{CRBN} substrate specificity, leading to the recruitment, ubiquitination and subsequent proteasomal degradation of neo-substrates such as Ikaros (IKZF1), Aiolos (IKZF3), Casein Kinase 1 alpha (CK1a or CSNK1A), G₁ to S phase transition protein 1 (GSPT1) and zinc finger protein 91 (ZFP91)⁶.

Neo-substrates interact with the degrader-bound surface of CRBN through recognition motifs known as degrons, characterised by a common β-hairpin loop with a conserved glycine at the apex, which is crucial for the interaction of the degron with the degrader compound. The amino acid sequences adjacent to the sentinel glycine in the β-hairpin loop are variable among neo-substrates and are critical for the specificity of recruitment and degradation of neo-substrates by a particular CRBN binding compound. In the case of the neo-substrates Ikaros, Aiolos and ZFP91, the degron motif is located within a C2H2 zinc finger (ZF) domain, with the first half of the domain harbouring a CxxCG sequence signature containing the conserved glycine just after the second Cysteine of the ZF^{7,8}.

The concept of a molecular switch for regulation of chimeric antigen receptor (CAR) expression is well recognised in the field as a desirable solution for enhanced safety and efficacy of CAR-T cells. In proof of concept work, Mackall's group at Stanford demonstrated that inducing degradation of a CAR in human T cells has two major advantages over conventional CAR expression in terms of CAR-T efficacy⁹. Firstly, degradation of the CAR during T cell manufacturing decreases T cell exhaustion associated with CAR tonic signalling and results in a T cell product that has enhanced T cell survival and leads to improvement of survival in tumour-bearing animals following its administration. Secondly, if drugs inducing degradation of the CAR on T cells can be administered to tumour bearing-animals in an intermittent dosing schedule, it allows transient rest of the T cells from antigen-induced activation in the tumour environment. Consequently the T cells can recover from exhaustion and, on re-expression of the CAR, have enhanced effector function. A third potential advantage of a degradation switch is the ability to switch off CAR-T function in the face of toxicity, providing a novel additional safety feature.

These data on the functional benefits of transient switching off of CAR in T cells were demonstrated using FK506 binding protein 12 destabilisation domain which can be blocked by a chemical probe⁹. For the technology to be translatable for clinical benefit, it would be necessary to develop CAR that are reversibly degradable in response to a drug that is approved for human use, functions at a pharmacologically attainable dose that is free of toxicity and has good penetration into tumour tissues.

Such proof-of-concept data has been provided by Jan and co-workers¹⁰ who have evaluated the molecular glue concept whereby IMiD drugs (thalidomide derivatives) bring together the CRL4^{CRBN} E3 ubiquitin ligase with known substrate proteins containing zinc finger degron motifs. A prototypic zinc finger-based 60 amino acid degron which was a hybrid of ZFP91 and IKZF3, and which they termed “super-degron”, was identified and shown to lead to lenalidomide-dependent reversible degradation of second generation (41 BB-zeta) chimeric antigen receptors tagged with SuperDegron at the cytoplasmic C-terminus.

There is a need for an improved degron technology to avoid CAR T cell toxicity and improve cancer treatment.

### SUMMARY OF THE INVENTION

The inventors have developed new drug-inducible degradation tags. Using structure and sequence analysis they generated 23 distinct protein tags (potential degrons) that were based on a chimeric structure of IKZF1 (Ikaros) and ZFP91 (Zinc Finger Protein 91). Drug-inducible degradation of each tag was determined and DCD23 (also referred to as iTAG1 herein) was identified as a potential degron candidate due to its relatively small size (60 amino acids) and good IMiD/CELMoDs degradation profile (FIG. 1). The inventors then made further amino acid substitutions to optimise the DCD23 degron sequence further. During this process, the inventors identified four core amino acid substitutions that disrupted nuclear localisation of the mutated degron tag (see iTAG2v1, iTAG2v2, and iTAG2 (note that the latter is also referred to as DCD23mut herein), which are described in more detail below). Disruption of nuclear localisation/accumulation in the cell nucleus is particularly advantageous when a degron tag is fused with a cell surface protein e.g. a chimeric receptor antigen (CAR), as it facilitates cell surface expression of the tagged protein.

The invention is based on the surprising finding that when a new mutated degron tag (such as DCD23mut, also referred to as iTAG2 herein) is fused to a CAR, the resultant CAR fusion protein has improved membrane stability compared to CAR tagged with DCD23 in Jurkat cells, 293T cells, and human primary T cells, whilst still being rapidly degraded in the presence of IMiDs/CELMoDs such as lenalidomide, pomalidomide and iberdomide. Advantageously, expression of DCD23mut-tagged CAR-T is also superior to that reported for a patented degron “SuperDegron” (WO2019089592). Furthermore, DCD23mut-tagged CAR-T expressed in primary human T cells show the same level of antigen-dependent reactivity as observed with untagged CAR-T (which is approximately 10-fold superior to the DCD23-tagged CAR-T).

Accordingly, the invention provides a degron tag comprising an amino acid sequence of LQCEICGFTCX₁QX₂GNLLX₃HIX₄LH (SEQ ID NO:66) wherein X₁, X₂, X₃ and, X₄ are not K, R or H.

Suitably, the degron tag may comprise an amino acid sequence of LQCEICGFTCX₁QX₂GNLLX₃HIX₄LH (SEQ ID NO:2) wherein:
X₁ is E or a conservative amino acid substitution thereof;
X₂ is A or a conservative amino acid substitution thereof;
X₃ is N or a conservative amino acid substitution thereof; and
X₄ is E or a conservative amino acid substitution thereof.

Suitably, the tag may comprise the amino acid sequence of LQCEICGFTCEQAGNLLNHIELH (SEQ ID NO:5).

Suitably, the tag may comprise the amino acid sequence of

(SEQ ID NO: 12)
LQCEICGFTCEQAGNLLNHIELHSG
or
(SEQ ID NO: 13)
LQCEICGFTCEQAGNLLNHIELHTG.

Suitably, the tag may comprise an additional N-terminal zinc finger alpha-helix subdomain and an additional C-terminal zinc finger beta-hairpin subdomain flanking the amino acid sequence of any one of SEQ ID NOs: 66, 2, 5, 12, or 13.

Suitably, the additional C-terminal zinc finger beta-hairpin subdomain may comprise the amino acid sequence CHLCNYACR (SEQ ID NO: 14) CHLCNYACQ (SEQ ID NO: 15), CHLCNYACRRRDAL (SEQ ID NO: 69) or CHLCNYACQRRDAL (SEQ ID NO: 70).

Suitably, the additional N-terminal zinc finger alpha-helix subdomain may comprise the amino acid sequence PNVLMVHX₅X₆SH (SEQ ID NO: 71) or FNVLMVHX₅X₆SH (SEQ ID NO:72); wherein X₅ and X₆ are not R, K or H.

Suitably, the additional N-terminal zinc finger alpha-helix subdomain may comprise the amino acid sequence PNVLMVHX₅X₆SH (SEQ ID NO: 16) or FNVLMVHX₅X₆SH (SEQ ID NO:17); wherein
X₅ is N or a conservative amino acid substitution thereof; and
X₆ is E or a conservative amino acid substitution thereof.

Suitably, the additional N-terminal zinc finger alpha-helix subdomain may comprise the amino acid sequence PNVLMVHNESH (SEQ ID NO: 22) or FNVLMVHNESH (SEQ ID NO: 23).

Suitably, the tag may comprise the amino acid sequence:

(SEQ ID NO: 73)
PNVLMVHX₅X₆SHTGEX₇PLQCEICGFTCX₁QX₂GNLLX₃HIX₄
LHSGEX₈PFKCHLCNYACRRRDAL,
or
(SEQ ID NO: 74)
FNVLMVHX₅X₆SHTGEX₇PLQCEICGFTCX₁QX₂GNLLX₃HIX₄
LHTGEX₈PFKCHLCNYACQRRDAL;

wherein X₇, and κ₈ are not R, K or H.

Suitably, the tag may comprise the amino acid sequence:

(SEQ ID NO: 24)
PNVLMVHX₅X₆SHTGEX₇PLQCEICGFTCX₁QX₂GNLLX₃HIX₄
LHSGEX₈PFKCHLCNYACRRRDAL,
or
(SEQ ID NO: 45)
FNVLMVHX₅X₆SHTGEX₇PLQCEICGFTCX₁QX₂GNLLX₃HIX₄
LHTGEX₈PFKCHLCNYACQRRDAL;

wherein:
X₇ is I or a conservative amino acid substitution thereof; and
X₈ is I or a conservative amino acid substitution thereof.

Suitably, the tag may comprise the amino acid sequence:

(SEQ ID NO: 43)
PNVLMVHNESHTGEIPLQCEICGFTCEQAGNLLNHIELHSGEIP
FKCHLCNYACRRRDAL,
or
(SEQ ID NO: 44)
FNVLMVHNESHTGEIPLQCEICGFTCEQAGNLLNHIELHTGEIP
FKCHLCNYACQRRDAL.

Suitably, the tag may have a length of about 23 to about 70 amino acids.

Suitably, the tag may have a length of about 23 to about 60 amino acids.

The invention also provides a fusion protein comprising a protein of interest and at least one degron tag of the invention.

Suitably, the degron tag may be located C-terminal to the protein of interest.

Suitably, the protein of interest may be a chimeric antigen receptor (CAR), a T cell receptor (TCR), a T cell receptor (TCR) fusion construct (TRuC), a T cell antigen coupler (TAC), a chimeric autoantibody receptor (CAAR), or an antibody-coupled T cell receptor (ACTR).

Suitably, the CAR fusion protein may comprise from N-terminus to C-terminus:
a) an extracellular ligand binding domain;
b) a transmembrane domain;
c) a cytoplasmic domain comprising at least one intracellular signaling domain; and
d) the at least one degron tag of the invention.

Suitably, the CAR fusion protein may comprise an extracellular ligand binding domain comprising an antibody or antigen binding fragment that is an scFv that binds to B7H3.

Suitably, the CAR fusion protein may comprise a CD28 transmembrane domain.

Suitably, the CAR fusion protein may comprise a CD3 signaling domain.

Suitably, the CAR fusion protein may comprise a CD28 co-stimulatory domain.

Suitably, the CAR fusion protein may comprise an extracellular ligand binding domain comprising an antibody or antigen binding fragment that is an scFv that binds to B7H3, a CD8 transmembrane domain, a CD3 signaling domain, a CD28 co-stimulatory domain and the at least one degron tag of the invention.

The invention also provides a non-naturally occurring nucleic acid sequence encoding a degron tag of the invention or a fusion protein of the invention.

The invention also provides a vector comprising a nucleic acid sequence of the invention.

Suitably, the vector may be a viral vector, optionally wherein the viral vector is selected from the group consisting of: a retroviral vector, an adenoviral vector, an adeno-associated viral vector, a herpes simplex viral vector, a vaccinia viral vector, a picornaviral vector, and an alphaviral vector.

The invention also provides a cell which expresses a nucleic acid sequence of the invention, or a vector of the invention.

Suitably, the cell may be an immune effector cell.

Suitably, the cell may be selected from the group consisting of: a T cell, B cell, plasma cell, NK cell, NKT cell, innate lymphoid cell, macrophage, dendritic cell, monocyte, neutrophil, basophil, eosinophil, mast cell, hematopoietic progenitor cell, hematopoietic stem cell, other adult stem cell such as neural, cornea, muscle, skin, small intestine, colon, bone, mesenchyme, embryonic stem cell and an induced pluripotent stem cell.

Suitably, the cell may be a mammalian cell, optionally wherein the cell is a human cell.

The invention also provides a pharmaceutical composition comprising a degron tag, fusion protein, nucleotide sequence, vector, or cell of the invention, and a pharmaceutically acceptable excipient, carrier, adjuvant and/or diluent.

The invention also provides a pharmaceutical composition of the invention for use as a medicament.

Suitably, the pharmaceutical composition of the invention may be for use in immune cell therapy.

The invention also provides a method of degrading a protein of interest comprising: contacting a cell in vitro or in vivo with an effective amount of an immunomodulatory drug (IMiD) or a cereblon modulator (CELMoD), wherein the cell expresses a nucleic acid encoding a fusion protein of the invention.

The invention also provides a method of degrading a protein of interest comprising: administering an effective amount of an immunomodulatory drug (IMiD) or a cereblon modulator (CELMoD) to a subject, wherein the subject has previously been treated via gene therapy causing at least some endogenous cells to express a nucleic acid encoding a fusion protein of the invention.

Suitably, the IMiD or CELMoD may be thalidomide, pomalidomide, lenalidomide, CC-122, CC-220 or CC-885.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Various aspects of the invention are described in further detail below.

### BRIEF DESCRIPTION OF THE FIGURES

Embodiments of the invention are further described hereinafter with reference to the accompanying drawings, in which:

FIG. 1: Experimental evaluation of the DCD tags. (a) Heat map summary of the degradation matrix data of all the DCDs tested in flow cytometry experiments. Normalized values were represented in the Log 2 Fold Change relative to the DMSO control. In the group of chimeric ZF DCDs (21-23), DCD21 led to the most potent degradation but had a significantly larger size compared to DCD23 (131 residues vs 60 residues, respectively). (b) Representation of iTAG1-DCD23 (60 aa). The 2 Cys and 2 His from the C2H2 zinc finger motif are highlighted in light grey. The crucial Glycine in the b-hairpin loop degron is highlighted in bold dark grey.

FIG. 2: Design of iTAG2. Representation of iTAG1(DCD23) and iTAG2. The 2 Cys and 2 His from the C2H2 zinc finger motif are highlighted. The crucial Glycine in the b-hairpin loop degron is highlighted. Mutated amino acids in iTAG2 are also highlighted in bold dark grey and underlined (and described in detail elsewhere herein). The sequence of SuperDegron (as described in WO2021188286 and WO2019089592) is also shown. The differences between the SuperDegron and iTAG are highlighted in italic on the SuperDegron sequence

FIG. 3: Experimental validation of iTAG2. (a) HMEC expressing dox inducible EGFP-iTAG1/2 or their corresponding P2A control treated with increasing concentrations of CC-220 for 4 h. Loss of target protein was validated via measuring EGFP median intensity by flow cytometry. The P2A is a ribosomal skip sequence that enables the separate translation of EGFP and iTAG, which provides evidence that the degradation is specific to the iTAG fusion. (b) Immunofluorescence images of HMEC expressing dox inducible EGFP-iTAG1/2 showing nuclear accumulation of iTAG1 that is abolished using iTAG2.

FIG. 4: iTAG2 variations. Representation of iTAG2 and its variations. The 2 Cys and 2 His from the C2H2 zinc finger motif are highlighted. The crucial Glycine in the b-hairpin loop degron is highlighted. Mutated amino acids are also highlighted in bold dark grey and underlined (and described in detail elsewhere herein).

FIG. 5: Constructs used for expression of tagged chimeric antigen receptors. CARs were expressed from a gamma retroviral construct SFG driven by EF1-alpha promoter. The RQR8 marker gene expresses a human CD34 epitope that allows for detection of transduced cells using the anti CD34 monoclonal antibody clone QBEND10. RQR8 is separated from the CARs by T2A ribosome skip site to allow equal translation of the two protein products. The chimeric antigen receptors comprised the TE9 anti B7H3 binder in ScFv format, CD28 and CD3zeta second generation signalling domains, and CD8 hinge and transmembrane sequences. At the C terminus, iTAG1(DCD23) or iTAG2 were cloned in frame with CD3zeta sequence.

FIG. 6. Flow cytometry histograms showing paired samples for expression of CAR (upper row) and CD34 (lower row) in transduced 293T (left) and Jurkat cells (right). The left-hand histograms represent the isotype control whilst right sided histograms are expression of CAR or CD34 respectively with numbers shown indicating the percentages of the population staining for the respective markers.

FIG. 7. Time course of downregulation of iTAG2 and DCD23-tagged chimeric antigen receptor (TE9-28-Z) following addition of IMiD drugs to transduced Jurkat cells. CAR expression was determined by direct staining of the TE9 ScFv component and evaluated by flow cytometry.

FIG. 8. Representative flow cytometry of human primary T cells directly stained for expression of the CD34 marker gene or the TE9-28-Z CAR following 24 hours culture in the presence or absence of 10 M iberdomide. The rectangle delineates the CAR-bright population that is effectively eliminated by addition of the drug. Flow plots are representative of two independent donors.

FIG. 9. CAR transduced human primary T cells from two independent donors were cultured at 1:1 effector to target ratio with SupT1 cells transduced to express human B7H3. Error bars indicate mean and standard deviation of duplicate values from two independent donors. UT=untransduced control, DCD2 tag and iTAG2 tag refers to TE9-28-Z CARs with the respective C terminus tags

FIG. 10. Comparisons of expression of iTAG2 tagged CAR with the non-mutated versions following transduction into Jurkat cells.

FIG. 11. Schematic representation of the Aiolos peptide-based TR-FRET assay used to measure the relative affinities of iTAG1 and iTAG2 for the complex formed between CRBN/DDB1 complex and various IMiDs.

FIG. 12. TR-FRET assay results obtained for iTAG1 and iTAG2 in the presence of lenalidomide, pomalidomide and iberdomide, including the IC₅₀s calculated from the TR-FRET curves.

FIG. 13. HMEC cells transiently transfected with GFP-superdegron fusion show strong nuclear GFP localisation by fluorescence microscopy. The GFP-iTAG1 and GFP-iTAG2 images are shown for comparison and are reproduced from FIG. 3b.

FIG. 14. A-Size exclusion chromatography (SEC) profiles obtained with samples composed of a mix of CRBN/DDB1 complex and iTAG2, in the presence or absence of iberdomide. B-SDS-PAGE analysis of representative fractions of the SEC analysis in the absence of iberdomide. The peak 1, corresponding to the higher molecular weight species, does not contain iTAG2, which is only present in peak 2. C-SDS-PAGE analysis of representative fractions of the SEC analysis in the presence of iberdomide. The peak 1, corresponding to the higher molecular weight species, contains iTAG2, confirming the formation of a complex between CRBN/DDB1 and iTAG2 in the presence of iberdomide.

FIG. 15. iTAG2 CAR-T are re-upregulated after IMiD washout. (a) γ-Retrovirally-transduced TE9 only or TE9-iTAG2 CAR-T cells were treated overnight with a range of iberdomide concentrations, and then analysed using flow cytometry for CAR expression. (b) CAR-T were than washed in PBS to remove drug, and rested for a further 24 h in RPMI1640 media. CAR expression was measured before and after the rest. 0.01 uM ibderdomide (shown with the black arrow) was identified as the optimal IMiD concentration to mediate CAR downregulation upon treatment and CAR re-upregulation upon withdrawal of drug. Representative data shown from one donor's CAR-T.

FIG. 16. iTAG2 CAR-T are functional in lentiviral format. (a) TE9-iTAG2 anti-B7H3 CAR-T constructs were originally expressed using γ-retroviral SFG constructs (Constructs 1 and 2). The TE9-iTAG2 transgene was then transferred to a pCCL lentiviral backbone (Construct 3) and, finally, codon optimized and streamlined by removing previously used molecular cloning restriction enzyme cleavage sites to produce Construct 4. The full expression cassette for Constructs 3 and 4 are illustrated in panel (b). (c) Constructs 3 and 4 were compared for their sensitivity to overnight 0.01 uM iberdomide treatment (data shown for technical replicates of three different CAR-T donors). Both Constructs 3 and 4 (C3 and C4) downregulated CAR in response to IMiD but, interestingly, Construct 3 expressed lower levels of RQR8 even in the absence of drug treatment. For this reason, Construct 4 was taken forward as the lead lentiviral vector for additional functional evaluation. (d) Construct 4 TE9-iTAG2 CAR-T cells were co-cultured overnight with SupT1-B7H3 T lymphoma target cells overnight at a 1:2 E:T ratio (data shown for six different CAR-T donors). Since complete target killing by CAR-T cells was observed, the E:T ratio was increased to 1:10, whereby ˜80% target killing was observed overnight. This indicated that TE9-iTAG2 CAR-T cells were highly cytotoxic and capable of potent serial target killing. (e, f) To confirm a lack of antigen non-specific TE9-iTAG2 CAR-T responsiveness, cytokine production was measured by ELISA after overnight culture either with SupT1-B7H3 targets at a 1:10 E:T ratio or with no targets. Cytokines were produced only in the presence of tumour targets (data shown for three different CAR-T donors). (g) Lack of codon-optimized TE9-iTAG2 cytotoxicity against B7H3-negative SupT1 targets was confirmed using an overnight luminescence-based assay at a 1:2 E:T ratio (data shown for three independent CAR-T donors).

The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

Various aspects of the invention are described in further detail below.

### DETAILED DESCRIPTION

The present invention provides novel drug-inducible degradation tags (degron tags), as well as fusion proteins, cells and compositions comprising the same. Methods of using the novel degrons, fusion proteins, cells, and compositions are also provided herein, including methods for modulating protein abundance in a target-specific manner via a degron tag. The invention may target endogenous and exogenous (e.g., therapeutic) proteins alike. As disclosed herein, degron tags are peptides that when fused to a target protein of interest (POI), transform the POI into a substrate that can be targeted for degradation.

Degron tags are also referred to as zinc finger degradation domains in the art. The terms “degron tag” and “zinc finger degradation domain” are used interchangeably herein. Degron tags as provided herein bind a complex formed between CRBN and an IMiD; or between CRBN and a cereblon modulator (CELMoD). In other words, a degron tag as provided herein binds a CRBN-IMiD or CRBN-CELMoD complex. Methods for determining binding of the degron tag to a CRBN-IMiD or CRBN-CELMoD complex are well known in the art, see for example Degorce et al., 2009 (PMID 20161833) on in vitro HTRF assays and Sievers et al 2018 (PMID 30385546) on using TR-FRET for ZF degron.

The degron tags provided herein comprise an amino acid sequence that is a mutated form of the minimal degron sequence (LQCEICGFTCRQKGNLLRHIKLH (SEQ ID NO:1)) that is provided in WO2021/188286 (where the underline denotes the amino acids that are mutated in the present invention). This minimal degron sequence corresponds to a full C2H2-type zinc finger protein domain, comprised of a N-terminal beta-hairpin subdomain containing 2 Cys and a C-terminal alpha-helix subdomain containing 2 His, with these 4 residues coordinating a single Zn2+ ion to stabilise the stereotypical C2H2 Zinc finger protein fold. The degron tags provided herein therefore comprise an amino acid sequence that is a mutated form of the minimal degron sequence shown to be functional in the prior art.

The inventors have shown herein that although the novel degron tags provided herein comprise an amino acid sequence that is a mutated variant of the minimal degron sequence shown to be functional previously, the mutated variants provided herein retain functionality (in terms of their capability to induce degradation of a fusion protein that comprises the degron tag), whilst also providing some surprising additional advantageous properties (such as increased membrane stability, and/or increased cell surface expression, of a fused cell surface protein, such as a CAR).

The inventors have exemplified the invention with novel degron tags that comprise four amino acid mutations in the minimal functional degron sequence that was previously identified. A degron tag is therefore provided herein, comprising an amino acid sequence of LQCEICGFTCX₁QX₂GNLLX₃HIX₄LH (SEQ ID NO:66) wherein X₁, X₂, X₃, and X₄ are not R, K or H.

In one example, a degron tag is provided herein, comprising an amino acid sequence of LQCEICGFTCX₁QX₂GNLLX₃HIX₄LH (SEQ ID NO:2) wherein:
X₁ is E or a conservative amino acid substitution thereof;
X₂ is A or a conservative amino acid substitution thereof;
X₃ is N or a conservative amino acid substitution thereof; and
X₄ is E or a conservative amino acid substitution thereof.

The inventors have shown that by mutating the amino acids X₁, X₂, X₃ and X₄ from basic amino acids to acidic, neutral or aliphatic amino acids it is possible to disrupt nuclear localisation (also referred to as reducing accumulation in the nucleus of the cell) of a degron-tagged fusion protein. Although the inventors have exemplified the invention by using a degron in which X, is E, X₂ is A, X₃ is N and X₄ is E, the invention is equally applicable to other degron variants in which the basic amino acids at positions X₁, X₂, X₃ and X₄ are substituted with an appropriate alternative non-basic amino acid. Accordingly, novel degron tags that disrupt nuclear localisation of a degron tagged fusion protein are provided herein, comprising an amino acid sequence of LQCEICGFTCX₁QX₂GNLLX₃HIX₄LH (SEQ ID NO:66), wherein X₁ is E or any non-basic amino acid substitution of E, X₂ is A or any non-basic amino acid substitution of A, X₃ is N or any non-basic amino acid substitution of N, and X₄ is E or any non-basic amino acid substitution of E.

In some examples, the non-basic amino acid substitution may be a conservative amino acid substitution of the variant amino acids exemplified herein. As would be known to a person of skill in the art, a “conservative amino acid substitution” refers to an amino acid replacement in a protein that changes a given amino acid to a different amino acid with similar biochemical properties, for example charge, hydrophobicity and size. Conservative amino acid substitutions produce a silent change and result in a functionally equivalent degron (in terms of its capability to bind a CRBN-IMiD complex, or a CRBN-CELMOD complex and induce degradation of a fusion protein that comprises the degron tag). Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

TABLE 1
examples of groups of amino acids with similar biochemical properties,
for example charge, hydrophobicity and size (and thus can be used
interchangeably at each mutation site provided herein).

| | | |
|---|---|---|
| ALIPHATIC | Non-polar | G A P |
| | | I L V |
| | Polar - uncharged | C S T M |
| | | N Q |
| | Polar - charged | D E |
| | | K R H |
| AROMATIC | | F W Y |

Accordingly, a degron tag of the invention may comprise an amino acid sequence of LQCEICGFTCX₁QX₂GNLLX₃HIX₄LH (SEQ ID NO: 3) wherein:
X₁ is E or D;
X₂ is A, G, P, I, L or V;
X₃ is N, Q, C, S, T or M; and
X₄ is E or D.

In one example, a degron tag of the invention may comprise an amino acid sequence of LQCEICGFTCX₁QX₂GNLLX₃HIX₄LH (SEQ ID NO: 4) wherein:
X₁ is E or D;
X₂ is A, G, or P;
X₃ is N, or Q; and
X₄ is E or D.

In one example, X₁ is E.

In one example, X₂ is A.

In one example X₃ is N.

In one example X₄ is E.

In one example, the degron tag comprises the amino acid sequence LQCEICGFTCEQAGNLLNHIELH (SEQ ID NO: 5). SEQ ID NO:5 is a sequence that is present in iTAG2, iTAGv1, and iTAGv2 that are described herein (corresponding to the core region of the mutated ZFP91 ZF2 beta-hairpin subdomain and IKAROS ZF2 alpha-helix subdomain sequences of these degron tags). It is also the equivalent mutated sequence of SuperDegron (i.e. the core region of the mutated variant of the ZFP91 ZF2 beta-hairpin subdomain and AIOLOS ZF2 alpha-helix subdomain sequences of SuperDegron). A degron comprising the amino acid sequence of SEQ ID NO: 5 may therefore be a degron based on an iTAG1 or SuperDegron amino acid sequence, wherein at least four mutations have been introduced (X₁, X₂, X₃ and X₄ as described above).

Suitably, the presence of an amino acid sequence of SEQ ID NO:66 or any one of SEQ ID NOs: 2 to 5 in the degron tag disrupts nuclear localization of a fusion protein that comprises the degron tag (e.g. a degron-tagged CAR). In other words, the presence of the amino acid sequence of SEQ ID NO:66 or any one of SEQ ID NOs: 2 to 5 in the degron tag reduces nuclear localization of a fusion protein comprising the degron tag compared to a fusion protein comprising an equivalent degron tag wherein the amino acid sequence of SEQ ID NO:66 or any one of SEQ ID NOs: 2 to 5 is replaced with the amino acid sequence LQCEICGFTCRQKGNLLRHIKLH (SEQ ID NO: 1). In this context, “reduce” may refer to a decrease or reduction of at least 5%, at least 10%, at least 20%, at least 30%, at least 50%, or more. As would be clear to a person of skill in the art, nuclear localization refers to being present (located) in the nucleus of a cell. Several methods are known in the art for determining nuclear localization, for example immunofluorescence, as used in the examples section below.

The degron tags provided herein may include additional amino acids (e.g. N-terminal and/or C-terminal to the amino acid sequence shown in SEQ ID NO: 66, or any one of SEQ ID NOs: 2 to 5), provided that the degron tag retains its ability to bind to a CRBN-IMiD complex or a CRBN-CELMoD complex and induce degradation of a fusion protein comprising the degron tag. These additional amino acids may correspond to residues in the native zinc finger domains or be different provided that the degron tag maintains a zinc finger-like fold and exhibits the requisite binding properties as disclosed herein.

For example, the degron tags provided herein may comprise the sequence

(SEQ ID NO: 67)
LQCEICGFTCX₁QX₂GNLLX₃HIX₄LHSG,
or
(SEQ ID NO: 68)
LQCEICGFTCX₁QX₂GNLLX₃HIX₄LHTG,

wherein X₁, X₂, X₃, and X₄ are not R, K or H.

For example, the degron tags provided herein may comprise the sequence

(SEQ ID NO: 6)
LQCEICGFTCX₁QX₂GNLLX₃HIX₄LHSG,
or
(SEQ ID NO: 7)
LQCEICGFTCX₁QX₂GNLLX₃HIX₄LHTG,

wherein:
X₁ is E or a conservative amino acid substitution thereof;
X₂ is A or a conservative amino acid substitution thereof;
X₃ is N or a conservative amino acid substitution thereof; and
X₄ is E or a conservative amino acid substitution thereof.

In other words, the degron tag may comprise the amino acid sequence

(SEQ ID NO: 8)
LQCEICGFTCX₁QX₂GNLLX₃HIX₄LHSG,
or
(SEQ ID NO: 9)
LQCEICGFTCX₁QX₂GNLLX₃HIX₄LHTG,

wherein:
X₁ is E or D;
X₂ is A, G, P, I, L or V;
X₃ is N, Q, C, S, T or M; and
X₄ is E or D.

In one example, the degron tag of the invention may comprise the amino acid sequence

(SEQ ID NO: 10)
LQCEICGFTCX₁QX₂GNLLX₃HIX₄LHSG,
or
(SEQ ID NO: 11)
LQCEICGFTCX₁QX₂GNLLX₃HIX₄LHTG,

wherein:
X₁ is E or D;
X₂ is A, G, or P;
X₃ is N, or Q; and
X₄ is E or D.

In one example, X₁ is E.

In one example, X₂ is A.

In one example X₃ is N.

In one example X₄ is E.

In one example, the degron tag comprises the amino acid sequence LQCEICGFTCEQAGNLLNHIELHSG (SEQ ID NO: 12) or LQCEICGFTCEQAGNLLNHIELHTG (SEQ ID NO:13). In this context, SEQ ID NO: 12 is a sequence that is present in iTAG2, iTAGv1, and iTAGv2 as described herein (corresponding to the mutated ZFP91 ZF2 beta-hairpin subdomain and IKAROS ZF2 alpha-helix subdomain sequences of these degron tags). Similarly, SEQ ID NO:13 is the equivalent mutated sequence of SuperDegron (i.e. a mutated variant of the ZFP91 ZF2 beta-hairpin subdomain and AIOLOS ZF2 alpha-helix subdomain sequences of SuperDegron).

The degron tags provided herein may include additional amino acids (e.g. an additional N-terminal zinc finger alpha-helix subdomain and/or an additional C-terminal zinc finger beta-hairpin subdomain) flanking the amino acid sequence of any one of SEQ ID NOs: 2 to 13, or 66 to 68. For example, a degron tag of the invention may comprise an additional N-terminal zinc finger alpha-helix subdomain and an additional C-terminal zinc finger beta-hairpin subdomain flanking the amino acid sequence of any one of SEQ ID NOs: 2 to 13, or 66 to 68. Various naturally occurring proteins contain zinc finger regions (also known as zinc finger motifs) that include a beta-hairpin loop (referred to as a zinc finger beta-hairpin subdomain in the context of the degron tags described herein) and an alpha-helix region (referred to as a zinc finger alpha-helix subdomain in the context of the degron tags described herein). In some embodiments, the degron tag may include a first sequence derivable from or which is at least part of a first zinc finger region, and a second sequence derivable from or which is part of an alpha-helix region of a second zinc finger region. The first and second zinc finger regions may be the same or different, provided that the resultant degron tag binds CRBN-IMiD or CRBN-CELMoD. Suitable N-terminal zinc finger alpha-helix subdomains are well known in the art, including but not limited to the IKAROS ZF1 alpha-helix subdomain, and the AIOLOS ZF1 alpha-helix subdomain used in the examples below. For example, ZFP91, ZN276, ZN517, ZN653, ZN654, ZN787, or ZN827 N-terminal zinc finger alpha-helix subdomains may be used. Appropriate C-terminal zinc finger beta-hairpin subdomains are also well known in the art, including but not limited to the IKAROS ZF3 beta-hairpin subdomain, and the AIOLOS ZF1 ZF3 beta-hairpin subdomain used in the examples below. For example, ZFP91, ZN276, ZN517, ZN653, ZN654, ZN787, or ZN827 C-terminal zinc finger beta-hairpin subdomains may be used.

The term “flanking” as used herein refers to the relative positioning of features within the same amino acid sequence. In this context, it therefore refers to additional amino acids or subdomains being N-terminal to, and/or C-terminal to, (as appropriate) the core sequence of any one of SEQ ID NOs: 2 to 13, or 66 to 68. It is noted that the flanking sequences do not need to be directly adjacent to the core sequence of SEQ ID NOs: 2 to 13, or 66 to 68; there may in intervening amino acids in between.

In one example, the degron comprises an additional N-terminal zinc finger alpha-helix subdomain and an additional C-terminal zinc finger beta-hairpin subdomain flanking the amino acid sequence of SEQ ID NO: 5. In other words, the degron may comprise a configuration that is, in the N-terminal to C-terminal direction: zinc finger alpha-helix subdomain—SEQ ID NO: 5—zinc finger beta-hairpin subdomain (optionally with intervening amino acids in between the subdomains and the amino acid sequence of SEQ ID NO:5). In this example, the additional C-terminal zinc finger beta-hairpin subdomain may comprise the amino acid sequence CHLCNYACR (SEQ ID NO: 14) or CHLCNYACQ (SEQ ID NO: 15) (such as, for example, CHLCNYACRRRDAL (SEQ ID NO: 69) or CHLCNYACQRRDAL (SEQ ID NO: 70)). In this example, the additional N-terminal zinc finger alpha-helix subdomain may also comprise the amino acid sequence PNVLMVHX₅X₆SH (SEQ ID NO: 71) or FNVLMVHX₅X₆SH (SEQ ID NO:72); wherein X₅ and X₆ are not R, K or H. For example, the additional N-terminal zinc finger alpha-helix subdomain may also comprise the amino acid sequence PNVLMVHX₅X₆SH (SEQ ID NO: 16) or FNVLMVHX₅X₆SH (SEQ ID NO:17); wherein X₅ is N or a conservative amino acid substitution thereof; and X₆ is E or a conservative amino acid substitution thereof. In other words, the additional N-terminal zinc finger alpha-helix subdomain may also comprise the amino acid sequence PNVLMVHX₅X₆SH (SEQ ID NO: 18) or FNVLMVHX₅X₆SH (SEQ ID NO:19); wherein X₅ is N, Q, C, S, T or M; and X₆ is E, or D. In one example, the additional N-terminal zinc finger alpha-helix subdomain may comprise the amino acid sequence PNVLMVHX₅X₆SH (SEQ ID NO: 20) or FNVLMVHX₅X₆SH (SEQ ID NO:21); wherein X₅ is N or Q; and X₆ is E or D. In a particular example, the additional N-terminal zinc finger alpha-helix subdomain may comprise the amino acid sequence PNVLMVHNESH (SEQ ID NO: 22) or FNVLMVHNESH (SEQ ID NO: 23). For example, when the degron comprises an additional N-terminal zinc finger alpha-helix subdomain and an additional C-terminal zinc finger beta-hairpin subdomain flanking the amino acid sequence of SEQ ID NO: 5, the additional N-terminal zinc finger alpha-helix subdomain may comprise the amino acid sequence of SEQ ID NO: 22 or SEQ ID NO: 23; and the additional C-terminal zinc finger beta-hairpin subdomain may comprise the amino acid sequence of SEQ ID NO: 14 or SEQ ID NO: 15.

Accordingly, in one example, the degron tag comprises an additional N-terminal zinc finger alpha-helix subdomain and an additional C-terminal zinc finger beta-hairpin subdomain flanking the amino acid sequence of SEQ ID NO: 5, wherein the additional N-terminal zinc finger alpha-helix subdomain comprises the amino acid sequence of SEQ ID NO: 22 and the additional C-terminal zinc finger beta-hairpin subdomain comprises the amino acid sequence of SEQ ID NO: 14. This arrangement corresponds to the sequences present in iTAG2.

In another example, the degron tag comprises an additional N-terminal zinc finger alpha-helix subdomain and an additional C-terminal zinc finger beta-hairpin subdomain flanking the amino acid sequence of SEQ ID NO: 5, wherein the additional N-terminal zinc finger alpha-helix subdomain comprises the amino acid sequence of SEQ ID NO: 23 and the additional C-terminal zinc finger beta-hairpin subdomain comprises the amino acid sequence of SEQ ID NO: 15.

In one example, the degron comprises an additional N-terminal zinc finger alpha-helix subdomain and an additional C-terminal zinc finger beta-hairpin subdomain flanking the amino acid sequence of SEQ ID NO: 12. In other words, the degron may comprise a configuration that is, in the N-terminal to C-terminal direction (left to right): zinc finger alpha-helix subdomain—SEQ ID NO: 12—zinc finger beta-hairpin subdomain (optionally with intervening amino acids in between the subdomains and the amino acid sequence of SEQ ID NO:12). In this example, the additional C-terminal zinc finger beta-hairpin subdomain may comprise the amino acid sequence CHLCNYACR (SEQ ID NO: 14) or CHLCNYACQ (SEQ ID NO: 15) (such as, for example, CHLCNYACRRRDAL (SEQ ID NO: 69) or CHLCNYACQRRDAL (SEQ ID NO: 70)). In this example, the additional N-terminal zinc finger alpha-helix subdomain may also comprise the amino acid sequence PNVLMVHX₅X₆SH (SEQ ID NO: 71) or FNVLMVHX₅X₆SH (SEQ ID NO:72); wherein X₅ and X₆ are not R, K or H. For example, the additional N-terminal zinc finger alpha-helix subdomain may also comprise the amino acid sequence PNVLMVHX₅X₆SH (SEQ ID NO: 16) or FNVLMVHX₅X₆SH (SEQ ID NO:17); wherein X₅ is N or a conservative amino acid substitution thereof; and X₆ is E or a conservative amino acid substitution thereof. In other words, the additional N-terminal zinc finger alpha-helix subdomain may also comprise the amino acid sequence PNVLMVHX₅X₆SH (SEQ ID NO: 18) or FNVLMVHX₅X₆SH (SEQ ID NO:19); wherein X₅ is N, Q, C, S, T or M; and X₆ is E, or D. In one example, the additional N-terminal zinc finger alpha-helix subdomain may comprise the amino acid sequence PNVLMVHX₅X₆SH (SEQ ID NO: 20) or FNVLMVHX₅X₆SH (SEQ ID NO:21); wherein X₅ is N or Q; and X₆ is E or D. In a particular example, the additional N-terminal zinc finger alpha-helix subdomain may comprise the amino acid sequence PNVLMVHNESH (SEQ ID NO: 22) or FNVLMVHNESH (SEQ ID NO: 23). For example, when the degron comprises an additional N-terminal zinc finger alpha-helix subdomain and an additional C-terminal zinc finger beta-hairpin subdomain flanking the amino acid sequence of SEQ ID NO: 12, the additional N-terminal zinc finger alpha-helix subdomain may comprise the amino acid sequence of SEQ ID NO: 22 or SEQ ID NO: 23; and the additional C-terminal zinc finger beta-hairpin subdomain may comprise the amino acid sequence of SEQ ID NO: 14 or SEQ ID NO: 15.

Accordingly, in one example, the degron tag comprises an additional N-terminal zinc finger alpha-helix subdomain and an additional C-terminal zinc finger beta-hairpin subdomain flanking the amino acid sequence of SEQ ID NO: 12, wherein the additional N-terminal zinc finger alpha-helix subdomain comprises the amino acid sequence PNVLMVHNESH (SEQ ID NO: 22) and the additional C-terminal zinc finger beta-hairpin subdomain comprises the amino acid sequence CHLCNYACR (SEQ ID NO: 14). This arrangement corresponds to the sequences present in iTAG2.

In another example, the degron tag comprises an additional N-terminal zinc finger alpha-helix subdomain and an additional C-terminal zinc finger beta-hairpin subdomain flanking the amino acid sequence of SEQ ID NO: 12, wherein the additional N-terminal zinc finger alpha-helix subdomain comprises the amino acid sequence FNVLMVHNESH (SEQ ID NO: 23) and the additional C-terminal zinc finger beta-hairpin subdomain comprises the amino acid sequence CHLCNYACQ (SEQ ID NO: 15).

In one example, the degron comprises an additional N-terminal zinc finger alpha-helix subdomain and an additional C-terminal zinc finger beta-hairpin subdomain flanking the amino acid sequence of SEQ ID NO: 13. In other words, the degron may comprise a configuration that is, in the N-terminal to C-terminal direction (left to right): zinc finger alpha-helix subdomain—SEQ ID NO: 13—zinc finger beta-hairpin subdomain (optionally with intervening amino acids in between the subdomains and the amino acid sequence of SEQ ID NO:13). In this example, the additional C-terminal zinc finger beta-hairpin subdomain may comprise the amino acid sequence CHLCNYACR (SEQ ID NO: 14) or CHLCNYACQ (SEQ ID NO: 15) (such as, for example, CHLCNYACRRRDAL (SEQ ID NO: 69) or CHLCNYACQRRDAL (SEQ ID NO: 70)). In this example, the additional N-terminal zinc finger alpha-helix subdomain may also comprise the amino acid sequence PNVLMVHX₅X₆SH (SEQ ID NO: 71) or FNVLMVHX₅X₆SH (SEQ ID NO:72); wherein X₅ and X₆ are not R, K or H. In this example, the additional N-terminal zinc finger alpha-helix subdomain may also comprise the amino acid sequence PNVLMVHX₅X₆SH (SEQ ID NO: 16) or FNVLMVHX₅X₆SH (SEQ ID NO:17); wherein X₅ is N or a conservative amino acid substitution thereof; and X₆ is E or a conservative amino acid substitution thereof.

In otherwards, the additional N-terminal zinc finger alpha-helix subdomain may also comprise the amino acid sequence PNVLMVHX₅X₆SH (SEQ ID NO: 18) or FNVLMVHX₅X₆SH (SEQ ID NO:19); wherein X₅ is N, Q, C, S, T or M; and X₆ is E, or D. In one example, the additional N-terminal zinc finger alpha-helix subdomain may comprise the amino acid sequence PNVLMVHX₅X₆SH (SEQ ID NO: 20) or FNVLMVHX₅X₆SH (SEQ ID NO:21); wherein X₅ is N or Q; and X₆ is E or D. In a particular example, the additional N-terminal zinc finger alpha-helix subdomain may comprises the amino acid sequence PNVLMVHNESH (SEQ ID NO: 22) or FNVLMVHNESH (SEQ ID NO: 23). For example, when the degron comprises an additional N-terminal zinc finger alpha-helix subdomain and an additional C-terminal zinc finger beta-hairpin subdomain flanking the amino acid sequence of SEQ ID NO: 13, the additional N-terminal zinc finger alpha-helix subdomain may comprise the amino acid sequence of SEQ ID NO: 22 or SEQ ID NO: 23; and the additional C-terminal zinc finger beta-hairpin subdomain may comprise the amino acid sequence of SEQ ID NO: 14 or SEQ ID NO: 15.

Accordingly, in one example, the degron tag comprises an additional N-terminal zinc finger alpha-helix subdomain and an additional C-terminal zinc finger beta-hairpin subdomain flanking the amino acid sequence of SEQ ID NO: 13, wherein the additional N-terminal zinc finger alpha-helix subdomain comprises the amino acid sequence PNVLMVHNESH (SEQ ID NO: 22) and the additional C-terminal zinc finger beta-hairpin subdomain comprises the amino acid sequence CHLCNYACR (SEQ ID NO: 14).

In another example, the degron tag comprises an additional N-terminal zinc finger alpha-helix subdomain and an additional C-terminal zinc finger beta-hairpin subdomain flanking the amino acid sequence of SEQ ID NO: 13, wherein the additional N-terminal zinc finger alpha-helix subdomain comprises the amino acid sequence FNVLMVHNESH (SEQ ID NO: 23) and the additional C-terminal zinc finger beta-hairpin subdomain comprises the amino acid sequence CHLCNYACQ (SEQ ID NO: 15).

In one example, the degron tag comprises the amino acid sequence:
PNVLMVHX₅X₆SHTGEX₇PLQCEICGFTCX₁QX₂GNLLX₃HIX₄LHSGEX₈PFKCHLCNYACRRR DAL (SEQ ID NO:73), wherein:
X₁, X₂, X₃, X₄, X₅, X₆, X₇, and X₈ are not R, K or H.

In one example, the degron tag comprises the amino acid sequence:
PNVLMVHX₅X₆SHTGEX₇PLQCEICGFTCX₁QX₂GNLLX₃HIX₄LHSGEX₈PFKCHLCNYACRRR DAL (SEQ ID NO:24), wherein:
X₁ is E or a conservative amino acid substitution thereof;
X₂ is A or a conservative amino acid substitution thereof;
X₃ is N or a conservative amino acid substitution thereof;
X₄ is E or a conservative amino acid substitution thereof.
X₅ is N or a conservative amino acid substitution thereof;
X₆ is E or a conservative amino acid substitution thereof;
X₇ is I or a conservative amino acid substitution thereof; and
X₈ is I or a conservative amino acid substitution thereof.

In other words, the degron tag may comprise the amino acid sequence PNVLMVHX₅X₆SHTGEX₇PLQCEICGFTCX₁QX₂GNLLX₃HIX₄LHSGEX₈PFKCHLCNYACRRR DAL (SEQ ID NO:25), wherein:
X₁ is E or D;
X₂ is A, G, P, I, L or V;
X₃ is N, Q, C, S, T or M;
X₄ is E or D;
X₅ is N, Q, C, S, T or M;
X₆ is E or D;
X₇ is I, L, V, G, A or P; and
X₈ is I, L, V, G, A or P.

In one example, the degron tag comprises the amino acid sequence PNVLMVHX₅X₆SHTGEX₇PLQCEICGFTCX₁QX₂GNLLX₃HIX₄LHSGEX₈PFKCHLCNYACRRR DAL (SEQ ID NO: 26), wherein:
X₁ is E or D;
X₂ is A, G, or P;
X₃ is N, or Q;
X₄ is E or D;
X₅ is N or Q;
X₆ is E or D;
X₇ is I, L or V; and
X₈ is I, L or V.

In one example, X₁ is E.

In one example, X₂ is A.

In one example, X₃ is N.

In one example, X₄ is E.

In one example, X₅ is N.

In one example, X₆ is E.

In one example, X₇ is I.

In one example, X₈ is I.

In one example, the degron tag comprises the amino acid sequence of SEQ ID NO: 27. (see Table 2 below).

In one example, the degron tag comprises the amino acid sequence of SEQ ID NO: 29 (see Table 2 below).

In one example, the degron tag comprises the amino acid sequence of SEQ ID NO: 31 (see Table 2 below).

In one example, the degron tag comprises the amino acid sequence of SEQ ID NO: 33 (see Table 2 below).

In one example, the degron tag comprises the amino acid sequence of SEQ ID NO: 35 (see Table 2 below).

In one example, the degron tag comprises the amino acid sequence of SEQ ID NO: 37 (see Table 2 below).

In one example, the degron tag comprises the amino acid sequence of SEQ ID NO: 39 (see Table 2 below).

In one example, the degron tag comprises the amino acid sequence of SEQ ID NO: 41 (see Table 2 below).

In one example, the degron tag comprises the amino acid sequence of SEQ ID NO: 43 (see Table 2 below).

In one example, the degron tag comprises the amino acid sequence:
FNVLMVHX₅X₆SHTGEX₇PLQCEICGFTCX₁QX₂GNLLX₃HIX₄LHTGEX₈PFKCHLCNYACQRR DAL (SEQ ID NO:74), wherein: X₁, X₂, X₃, X₄, X₅, X₆, X₇, and X₈ are not R, K or H.

In one example, the degron tag comprises the amino acid sequence:
FNVLMVHX₅X₆SHTGEX₇PLQCEICGFTCX₁QX₂GNLLX₃HIX₄LHTGEX₈PFKCHLCNYACQRR DAL (SEQ ID NO:45), wherein:
X₁ is E or a conservative amino acid substitution thereof;
X₂ is A or a conservative amino acid substitution thereof;
X₃ is N or a conservative amino acid substitution thereof;
X₄ is E or a conservative amino acid substitution thereof.
X₅ is N or a conservative amino acid substitution thereof;
X₆ is E or a conservative amino acid substitution thereof;
X₇ is I or a conservative amino acid substitution thereof; and
X₈ is I or a conservative amino acid substitution thereof.

In other words, the degron tag may comprise the amino acid sequence FNVLMVHX₅X₆SHTGEX₇PLQCEICGFTCX₁QX₂GNLLX₃HIX₄LHTGEX₈PFKCHLCNYACQRR DAL (SEQ ID NO:46), wherein:
X₁ is E or D;
X₂ is A, G, P, I, L or V;
X₃ is N, Q, C, S, T or M;
X₄ is E or D;
X₅ is N, Q, C, S, T or M;
X₆ is E or D;
X₇ is I, L, V, G, A or P; and
X₈ is I, L, V, G, A or P.

In one example, the degron tag comprises the amino acid sequence FNVLMVHX₅X₆SHTGEX₇PLQCEICGFTCX₁QX₂GNLLX₃HIX₄LHTGEX₈PFKCHLCNYACQRR DAL (SEQ ID NO:47), wherein:
X₁ is E or D;
X₂ is A, G, or P;
X₃ is N, or Q;
X₄ is E or D;
X₅ is N or Q;
X₆ is E or D;
X₇ is I, L or V; and
X₈ is I, L or V.

In one example, X₁ is E.

In one example, X₂ is A.

In one example, X₃ is N.

In one example, X₄ is E.

In one example, X₅ is N.

In one example, X₆ is E.

In one example, X₇ is I.

In one example, X₈ is I.

In one example, the degron tag comprises the amino acid sequence of SEQ ID NO: 28 (see Table 2 below).

In one example, the degron tag comprises the amino acid sequence of SEQ ID NO: 30 (see Table 2 below).

In one example, the degron tag comprises the amino acid sequence of SEQ ID NO: 32 (see Table 2 below).

In one example, the degron tag comprises the amino acid sequence of SEQ ID NO: 34 (see Table 2 below).

In one example, the degron tag comprises the amino acid sequence of SEQ ID NO: 36 (see Table 2 below).

In one example, the degron tag comprises the amino acid sequence of SEQ ID NO: 38 (see Table 2 below).

In one example, the degron tag comprises the amino acid sequence of SEQ ID NO: 40 (see Table 2 below).

In one example, the degron tag comprises the amino acid sequence of SEQ ID NO: 42 (see Table 2 below).

In one example, the degron tag comprises the amino acid sequence of SEQ ID NO: 44 (see Table 2 below).

Particular amino acid sequences that may be comprised within a degron tag of the invention are listed in the table below, wherein the specific mutations identified herein are introduced into the corresponding iTAG1 and SuperDegron sequences described herein. As can be seen from the table, four specific mutations are contemplated within the minimal degron sequence, with optional (one, two, three or four) additional mutations contemplated in the flanking sequences. In each case, the mutation is underlined for ease of review and the minimal degron sequence has been italicised and is in bold.

TABLE 2
Examples of specific novel degron sequences that are contemplated herein

| Sequence details | Amino acid sequence |
|---|---|
| Minimal degron sequence of | LQCEICGFTCEQAGNLLNHIELH |
| WO2021/188286 plus 4 mutations (X₁, X₂, X₃, | (SEQ ID NO: 5) |
| X₄) described elsewhere herein | This sequence is present in iTAG2, iTAG2v1, |
| | iTAG2v2 described herein |
| Full ZFP91 ZF2 beta hairpin and IKAROS | LQCEICGFTCEQAGNLLNHIELH |
| ZF2 alpha helix of iTAG1 plus 4 mutations in | SG (SEQ ID NO: 12) |
| minimal degron sequence (X₁, X₂, X₃, X₄) | This sequence is present in iTAG2, iTAG2v1, |
| described elsewhere herein | iTAG2v2 described herein |
| Full ZFP91 ZF2 beta hairpin and AIOLOS | LQCEICGFTCEQAGNLLNHIELH |
| ZF2 alpha helix of SuperDegron plus 4 | TG (SEQ ID NO: 13) |
| mutations in minimal degron sequence (X₁, | |
| X₂, X₃, X₄) described elsewhere herein | |
| Full length iTAG1 plus 4 mutations in minimal | PNVLMVHKRSHTGERPLQCEICGFTCEQAGNLLNHIELH |
| degron sequence (X₁, X₂, X₃, X₄) described | SGEKPFKCHLCNYACRRRDAL |
| elsewhere herein | (SEQ ID NO: 27) This is the sequence of |
| | iTAG2v1 described herein |
| Full length SuperDegron plus 4 mutations in | FNVLMVHKRSHTGERPLQCEICGFTCEQAGNLLNHIELH |
| minimal degron sequence (X₁, X₂, X₃, X₄) | TGEKPFKCHLCNYACQRRDAL |
| described elsewhere herein | (SEQ ID NO: 28) |
| Full length iTAG1 plus 4 mutations in minimal | PNVLMVHKRSHTGEIPLQCEICGFTCEQAGNLLNHIELH |
| degron sequence (X₁, X₂, X₃, X₄) and 1 | SGEKPFKCHLCNYACRRRDAL |
| additional mutation (X₇) described elsewhere | (SEQ ID NO: 29) |
| herein | This is the sequence of |
| | iTAG2v2 described herein |
| Full length SuperDegron plus 4 mutations in | FNVLMVHKRSHTGEIPLQCEICGFTCEQAGNLLNHIELH |
| minimal degron sequence (X₁, X₂, X₃, X₄) and | TGEKPFKCHLCNYACQRRDAL |
| 1 additional mutation (X₇) described | (SEQ ID NO: 30) |
| elsewhere herein | |
| Full length iTAG1 plus 4 mutations in minimal | PNVLMVHKESHTGEIPLQCEICGFTCEQAGNLLNHIELH |
| degron sequence (X₁, X₂, X₃, X₄) and 2 | SGEKPFKCHLCNYACRRRDAL |
| additional mutations (X₇ and X₆) described | (SEQ ID NO: 31) |
| elsewhere herein | |
| Full length SuperDegron plus 4 mutations in | FNVLMVHKESHTGEIPLQCEICGFTCEQAGNLLNHIELH |
| minimal degron sequence (X₁, X₂, X₃, X₄) and | TGEKPFKCHLCNYACQRRDAL |
| 2 additional mutations (X₇ and X₆) described | (SEQ ID NO: 32) |
| elsewhere herein | |
| Full length iTAG1 plus 4 mutations in minimal | PNVLMVHNRSHTGEIPLQCEICGFTCEQAGNLLNHIELH |
| degron sequence (X₁, X₂, X₃, X₄) and 2 | SGEKPFKCHLCNYACRRRDAL |
| additional mutations (X₇ and X₅) described | (SEQ ID NO: 33) |
| elsewhere herein | |
| Full length SuperDegron plus 4 mutations in | FNVLMVHNRSHTGEIPLQCEICGFTCEQAGNLLNHIELH |
| minimal degron sequence (X₁, X₂, X₃, X₄) and | TGEKPFKCHLCNYACQRRDAL |
| 2 additional mutations (X₇ and X₅) described | (SEQ ID NO: 34) |
| elsewhere herein | |
| Full length iTAG1 plus 4 mutations in minimal | PNVLMVHKRSHTGEIPLQCEICGFTCEQAGNLLNHIELH |
| degron sequence (X₁, X₂, X₃, X₄) and 2 | SGEIPFKCHLCNYACRRRDAL |
| additional mutations (X₇ and X₈) described | (SEQ ID NO: 35) |
| elsewhere herein | |
| Full length SuperDegron plus 4 mutations in | FNVLMVHKRSHTGEIPLQCEICGFTCEQAGNLLNHIELH |
| minimal degron sequence (X₁, X₂, X₃, X₄) and | TGEIPFKCHLCNYACQRRDAL |
| 2 additional mutations (X₇ and X₈) described | (SEQ ID NO: 36) |
| elsewhere herein | |
| Full length iTAG1 plus 4 mutations in minimal | PNVLMVHNESHTGEIPLQCEICGFTCEQAGNLLNHIELH |
| degron sequence (X₁, X₂, X₃, X₄) and 3 | SGEKPFKCHLCNYACRRRDAL |
| additional mutations (X₇, X₆ and X₅) | (SEQ ID NO: 37) |
| described elsewhere herein | |
| Full length SuperDegron plus 4 mutations in | FNVLMVHNESHTGEIPLQCEICGFTCEQAGNLLNHIELH |
| minimal degron sequence (X₁, X₂, X₃, X₄) and | TGEKPFKCHLCNYACQRRDAL |
| 3 additional mutations (X₇, X₆ and X₅) | (SEQ ID NO: 38) |
| described elsewhere herein | |
| Full length iTAG1 plus 4 mutations in minimal | PNVLMVHKESHTGEIPLQCEICGFTCEQAGNLLNHIELH |
| degron sequence (X₁, X₂, X₃, X₄) and 3 | SGEIPFKCHLCNYACRRRDAL |
| additional mutations (X₇, X₆ and X₈) described | (SEQ ID NO: 39) |
| elsewhere herein | |
| Full length SuperDegron plus 4 mutations in | FNVLMVHKESHTGEIPLQCEICGFTCEQAGNLLNHIELH |
| minimal degron sequence (X₁, X₂, X₃, X₄) and | TGEIPFKCHLCNYACQRRDAL |
| 3 additional mutations (X₇, X₆ and X₈) | (SEQ ID NO: 40) |
| described elsewhere herein | |
| Full length iTAG1 plus 4 mutations in minimal | PNVLMVHNRSHTGEIPLQCEICGFTCEQAGNLLNHIELH |
| degron sequence (X₁, X₂, X₃, X₄) and 3 | SGEIPFKCHLCNYACRRRDAL |
| additional mutations (X₇, X₅ and X₈) | (SEQ ID NO: 41) |
| described elsewhere herein | |
| Full length SuperDegron plus 4 mutations in | FNVLMVHNRSHTGEIPLQCEICGFTCEQAGNLLNHIELH |
| minimal degron sequence (X₁, X₂, X₃, X₄) and | TGEIPFKCHLCNYACQRRDAL |
| 3 additional mutations (X₇, X₅ and X₈) | (SEQ ID NO: 42) |
| described elsewhere herein | |
| Full length iTAG1 plus 4 mutations in minimal | PNVLMVHNESHTGEIPLQCEICGFTCEQAGNLLNHIELH |
| degron sequence (X₁, X₂, X₃, X₄) and 4 | SGEIPFKCHLCNYACRRRDAL |
| additional mutations (X₅, X₆, X₇ and X₈) | (SEQ ID NO: 43) This is the sequence of |
| described elsewhere herein | iTAG2 described herein |
| Full length SuperDegron plus 4 mutations in | FNVLMVHNESHTGEIPLQCEICGFTCEQAGNLLNHIELH |
| minimal degron sequence (X₁, X₂, X₃, X₄) and | TGEIPFKCHLCNYACQRRDAL |
| 3 additional mutations (X₅, X₆, X₇ and X₈) | (SEQ ID NO: 44) |
| described elsewhere herein | |

The degron tags provided herein are generally described using the core amino acid sequence of any one of SEQ ID NOs: 2 to 13, or 66 to 68, which include four amino acid substitutions compared to the known minimal functional degron sequence of LQCEICGFTCRQKGNLLRHIKLH (where the underline denotes the amino acids that are mutated in the present invention). Although the degron tags provided herein are generally described with all four amino acid substitutions, it would be clear to a person of skill in the art, that degron tags with one or more, two or more, three or more of these mutations may also be contemplated herein.

Accordingly, a degron tag is therefore provided herein, comprising an amino acid sequence of LQCEICGFTCX₁QX₂GNLLX₃HIX₄LH (SEQ ID NO:75) wherein one, two or three of the following amino acid substitutions are made:
at position X₁, R is substituted with any amino acid other than R, K or H; and/or
at position X₂, K is substituted with any amino acid other than R, K or H; and/or
at position X₃, R is substituted with any amino acid other than R, K or H; and/or
at position X₄, K is substituted with any amino acid other than R, K or H.

In one example, a degron tag is provided herein, comprising an amino acid sequence of LQCEICGFTCX₁QX₂GNLLX₃HIX₄LH (SEQ ID NO:48) wherein one, two or three of the following amino acid substitutions are made:
at position X₁, R is substituted with E or a conservative amino acid substitution thereof; and/or
at position X₂, K is substituted with A or a conservative amino acid substitution thereof; and/or
at position X₃, R is substituted with N or a conservative amino acid substitution thereof; and/or
at position X₄, K is substituted with E or a conservative amino acid substitution thereof.

As would be clear to a person of skill in the art, at any positions where an amino acid substitution is not made, the original amino acid is retained (i.e. R at position X₁, K at position X₂, R at position X₃, K at position X₄ etc).

Accordingly, a degron tag as provided herein may comprise an amino acid sequence of LQCEICGFTCX₁QX₂GNLLX₃HIX₄LH (SEQ ID NO: 49) wherein one, two or three of the following amino acid substitutions are made:
at position X₁, R is substituted with E or D; and/or
at position X₂, K is substituted with A, G, P, I, L or V; and/or
at position X₃, R is substituted with N, Q, C, S, T or M; and/or
at position X₄, K is substituted with E or D.

In one example, a degron tag as provided herein may comprise an amino acid sequence of LQCEICGFTCX₁QX₂GNLLX₃HIX₄LH (SEQ ID NO: 50) wherein one, two or three of the following amino acid substitutions are made:
at position X₁, R is substituted with E or D; and/or
at position X₂, K is substituted with A, G, or P; and/or
at position X₃, R is substituted with N, or Q; and/or
at position X₄, K is substituted with E or D.

In one example, a degron tag as provided herein may therefore comprise an amino acid sequence of LQCEICGFTCX₁QX₂GNLLX₃HIX₄LH (SEQ ID NO: 51) wherein one, two or three of the following amino acid substitutions are made:
at position X₁, R is substituted with E; and/or
at position X₂, K is substituted with A; and/or
at position X₃, R is substituted with N; and/or
at position X₄, K is substituted with E.

Accordingly, in one example, a degron tag as provided herein may therefore comprise an amino acid sequence of LQCEICGFTCEQKGNLLRHIKLH (SEQ ID NO: 52), LQCEICGFTCRQAGNLLRHIKLH (SEQ ID NO: 53), LQCEICGFTCRQKGNLLNHIKLH (SEQ ID NO: 54), or LQCEICGFTCRQKGNLLRHIELH (SEQ ID NO: 55) (all of which have one of the substitutions set out above). Examples wherein the degron tag would have two, or three, of the recited substitutions are also contemplated herein and would be readily identifiable to a person of skill in the art. Examples of such tags include: LQCEICGFTCEQAGNLLRHIKLH (SEQ ID NO: 56); LQCEICGFTCEQKGNLLNHIKLH (SEQ ID NO: 57); LQCEICGFTCEQKGNLLNHIELH (SEQ ID NO: 58); LQCEICGFTCRQAGNLLNHIKLH (SEQ ID NO: 59); LQCEICGFTCRQAGNLLRHIELH (SEQ ID NO: 60); LQCEICGFTCRQKGNLLNHIELH (SEQ ID NO: 61); LQCEICGFTCEQAGNLLNHIKLH (SEQ ID NO: 62); LQCEICGFTCEQAGNLLRHIELH (SEQ ID NO: 63); LQCEICGFTCEQKGNLLNHIELH (SEQ ID NO: 64); or LQCEICGFTCRQAGNLLNHIELH (SEQ ID NO: 65). In each of these examples, the mutated amino acid(s) relative to SEQ ID NO:1 are underlined.

Degron tags of the present invention are peptides generally having about 23 amino acids to about 70 amino acids, typically about 23 amino acids to about 60 amino acids. In some examples, the degron tags of the invention are peptides generally having about 23 to about 30 amino acids. The terms “peptide”, “polypeptide”, and “protein” are used herein consistent with their art-recognized meanings.

Fusion proteins comprising a protein of interest (POI) and at least one degron tag of the invention are also provided herein. The degron tag may be used as a “safety switch” when linked to a therapeutic protein of interest (POI) as it can be used to target degradation of the POI in situations where POI expression is not desired. The ability to degrade a particular endogenous protein of interest by creating POI-degron tag fusions and administering an IMiD or CELMoD can be used to treat disorders wherein expression of a protein above certain threshold levels within the cell leads to a diseased state. Other applications of this technology include 1) targeted degradation of proteins where pathology is a function of gain of function mutation(s), 2) targeted degradation of proteins where pathology is a function of amplification or increased expression, 3) targeted degradation of proteins that are manifestations of monogenetic disease, 4) targeted degradation of proteins where genetic predisposition manifests over longer periods and often after alternative biological compensatory mechanisms are no longer adequate, for example, but not limited to, hypercholesterolemia and proteinopathies. In addition, POI-degron tag fusions can be used to evaluate the function of an endogenous protein or validate an endogenous protein as a target for therapy of a disease state. Accordingly, the degron tags of the present invention can be utilized to produce a stably expressed endogenous protein-degron tag fusion protein or exogenous protein-degron tag fusion protein. Endogenous proteins originate within an organism, tissue or cell and are expressed by that same organism, tissue or cell, whereas exogenous proteins originate outside of an organism, tissue or cell and are introduced into the organism, tissue or cell.

As stated elsewhere herein, the fusion proteins provided herein may comprise a degron of the invention fused to a protein of interest. The protein of interest may be an immune surface receptor and/or “chimeric immunomodulatory receptor”. The protein of interest may be selected from the group consisting of a chimeric antigen receptor (CAR), a T cell receptor (TCR), a T cell receptor (TCR) fusion construct (TRuC), a T cell antigen coupler (TAC), a chimeric autoantibody receptor (CAAR), or an antibody-coupled T cell receptor (ACTR).

The degrons of the invention are particularly useful when the POI is a chimeric antigen receptor (CAR) protein. Accordingly, the CAR-Degron Tag Fusion proteins are particularly provided herein. Genetically modified T cells expressing chimeric antigen receptors (CAR-T therapy) have shown to have therapeutic efficacy in a number of cancers, including lymphoma, chronic lymphocytic leukemia, acute lymphoblastic leukemia, and neuroblastoma. Two autologous CAR-T cell therapies (Kymriah™ and Yescarta™) have been approved by the FDA. In common, both are CD19-specific CAR-T cell therapies lysing CD19-positive targets (normal and malignant B lineage cells). CAR-T therapy is not, however, without significant side effects. Although most adverse events with CAR-T are tolerable and acceptable, the administration of CAR-T cells has, in a number of cases, resulted in severe systemic inflammatory reactions, including cytokine release syndrome (CRS) and tumor lysis syndrome. The dramatic clinical activity of CAR-T cell therapy presents a need to implement safety strategies to rapidly reverse or abort the T cell responses in patients undergoing CRS or associated adverse events.

Accordingly, the present invention includes fusion proteins that contain a CAR and at least one degron tag. The CARs are further characterized in that they include an extracellular ligand binding domain capable of binding to an antigen, a transmembrane domain, and an intracellular domain in this order from the N-terminal side, wherein the intracellular domain includes at least one signalling domain. The degron tag(s) can be located at the N-terminus or between the extracellular binding domain and the transmembrane domain, provided that there is no disruption to antigen binding or insertion into the membrane. Similarly, degron tag(s) can be located at the C-terminus, between the transmembrane domain and the intracellular domain or between signalling domains when more than one is present, provided that there is no disruption of intracellular signalling or insertion into the membrane. The degron tag is preferably located at the C-terminus. In other words, the degron is preferably at the 3′ end of the fusion protein, such that it is fused to the 3′ end of the C-terminal intracellular signalling domain in the cytoplasmic domain of the CAR.

In one embodiment, the fusion protein includes a CAR which is tisagenlecleucel (Kymriah™) and a degron tag as described herein. Tisagenlecleucel is genetically modified, antigen-specific, autologous T cells that target CD19. The extracellular domain of the CAR is a murine anti-CD19 single chain antibody fragment (scFv) from murine monoclonal FMC63 hybridoma. The intracellular domain of the CAR is a T cell signaling domain derived from human CD3z and a co-stimulatory domain derived from human 4-1BB (CD137). The transmembrane domain and a spacer, located between the scFv domain and the transmembrane domain, are derived from human CD8a. Kymriah™ (tisagenlecleucel) is approved for the treatment of patients up to 25 years of age with B-cell precursor acute lymphoblastic leukemia (ALL) that is refractory or in relapse (R/R) and for the treatment of adults with R/R diffuse large B-cell lymphoma (DLBCL), the most common form of non-Hodgkin's lymphoma, as well as high grade B-cell lymphoma and DLBCL arising from follicular lymphoma. The degron tag may be any of the degron tags disclosed herein.

In one embodiment, the fusion protein includes a CAR which is axicabtagene ciloleucel (Yescarta™) and a degron tag. Axicabtagene ciloleucel is genetically modified, antigen-specific, autologous T cells that target CD19. The extracellular domain of the CAR is a murine anti-CD19 single chain antibody fragment (scFv). The intracellular domain of the CAR is two signaling domains, one derived from human CD3z and one derived from human CD28. Yescarta™ (axicabtagene ciloleucel) is approved for the treatment of adults with R/R large B cell lymphoma including DLBCL not otherwise specified, primary mediastinal large B-cell lymphoma, high grade B-cell lymphoma, and DLBCL arising from follicular lymphoma. The degron tag may be any of the degron tags disclosed herein.

In one embodiment, the antigen binding moiety portion of the CAR is designed to treat a particular cancer. For example, a CAR designed to target CD19 can be used to treat cancers and disorders including pre-B ALL (paediatric indication), adult ALL, mantle cell lymphoma, diffuse large B-cell lymphoma, and salvage post allogenic bone marrow transplantation.

Further features of CAR proteins, nucleic acids encoding CAR proteins, immune effector cells expressing CARs and methods of using CAR expressing cells for the treatment of diseases are disclosed in U.S. Patent Application Publication 2018/0169109 A1, incorporated herein by reference.

As stated elsewhere herein, the fusion proteins provided herein may comprise a degron of the invention fused to a protein of interest. The protein of interest may be a T cell receptor (TCR). There are several different types of TCR that may be used in the context of the invention, for example, the TCR may be a chimeric T cell receptor, an artificial T cell receptor, or a synthetic T cell receptor. Furthermore, the TCR may be an antibody-coupled T cell receptor (ACTR), a T cell receptor fusion construct (TRuC), or a T cell antigen coupler (TAC).

In one example, the POI may be a T Cell Receptor Fusion Construct (TRuC). T cell receptor fusion constructs (TRuCs) are receptor proteins that include an antibody-based binding domain fused to T cell receptor (TCR) subunit designed for effective recognition of target cell surface antigens. TRuCs include a specific ligand antibody fused to the extracellular N-termini of a number of TCR subunits (for example TCRα, TCRβ, CD3ϵ, CD3γ and CD3δ). TRuCs provide target specificity and HLA-independent target cell elimination capabilities. TRuCs can be integrated into a native TCR complex on the surface of a cell, such as a T cell. Unlike CARs, TRuCs become a functional component of the TCR complex. TRuC-T cells demonstrate potent anti-tumor activity in both liquid and solid tumor xenograft models.

In another example, the POI may be an Antibody-coupled T cell receptor (ACTR). An ACTR is a non-naturally occurring molecule that can be expressed on the surface of a host cell and comprises an extracellular domain (e.g., a CD16A extracellular domain) capable of binding to a target molecule containing an Fc portion and one or more cytoplasmic signaling domains for triggering effector functions of the immune cell expressing the ACTR polypeptide, wherein at least two domains of the ACTR polypeptide may be derived from different molecules. The ACTR polypeptide may comprise a CD16A extracellular domain capable of binding to a target molecule containing an Fc portion, a transmembrane domain, one or more co-stimulatory signalling domains, and a CD3ζ cytoplasmic signalling domain. At least one of the co-stimulatory signalling domains may be a CD28 co-stimulatory domain. The ACTR polypeptide can either be free of a hinge domain from any non-CD 16A receptor or comprise more than one co-stimulatory signalling domain if the transmembrane domain is a CD8 transmembrane domain.

In another example, the POI may be a T Cell Antigen Coupler (TAC). The T cell antigen coupler (TAC) is a platform that co-opts the endogenous TCR with MHC-independent mechanisms to induce a more efficient target cell response and reduce toxicity. TAC chimeric proteins are coupled to a TCR to recognize an antigen via CD3 domain binding, resulting in a TCR/CD3 complex formation.

Alternatively, the antigen binding cell surface protein may be a Chimeric auto-antibody receptor (CAAR). CAARs are a modified form of CARs which identify cells secreting antibodies such as autoreactive B cells. CAARs include a specific antigen, a transmembrane domain, and an intracellular signalling domain with or without a co-stimulatory domain. CAARs recognize and bind to the target autoantibodies expressed on autoreactive cells via the specific antigen, and subsequently, destroy them.

Nucleic acid sequences and nucleic acid molecules that encode a degron or a fusion protein described herein are also provided. The nucleic acid sequences and nucleic acid molecules may be a non-naturally occurring nucleic acid sequence encoding a degron tag or fusion protein as described herein.

Vectors are also provided that comprise a nucleic acid sequence that encodes a novel degron or fusion protein described herein. A “vector” is a composition of matter which contains a nucleic acid and which can be used to deliver the nucleic acid to the interior of a cell. Numerous vectors are known in the art including linear polynucleotides, polynucleotides associated with ionic or amphiphilic compounds, plasmids and viruses. Thus, the term “vector” includes an autonomously replicating plasmid or a virus. The term should also be construed to include non-plasmid and non-viral compounds which facilitate transfer of nucleic acid into cells, such as, for example, polylysine compounds and liposomes or lipid nanoparticles. The vector may therefore be a non-viral vector (e.g. plasmids, polylysine compounds and liposomes or lipid nanoparticles) or a viral vector. Representative examples of viral vectors include: a retroviral vector, an adenoviral vector, an adeno-associated viral vector, a herpes simplex viral vector, a vaccinia viral vector, a picornaviral vector, and an alphaviral vector. An example of a retroviral vector is a lentiviral vector.

Vectors can be delivered in vivo by administration to an individual subject, typically by systemic administration (e.g., intravenous, intraperitoneal, intramuscular, intrathecal, intratracheal, subdermal, or intracranial infusion) or topical application. Alternatively, vectors can be delivered to cells ex vivo, such as cells explanted from an individual patient (e.g., lymphocytes, bone marrow aspirates or tissue biopsy) or universal donor hematopoietic stem cells, followed by reimplantation of the cells into a patient, usually after selection for cells which have incorporated the vector.

In certain embodiments, a nucleic acid encoding a degron tag can be genomically inserted in-frame with a gene encoding a protein that is involved in a disorder. Representative examples of particular genes involved in disorders that may be targeted for degron tag insertion include alpha-1 antitrypsin (A1AT), apolipoprotein B (apoB), angiopoietin-like protein 3 (ANGPTL3), proprotein convertase subtilisin/kexin type 9 (PCSK9), apolipoprotein C3 (APOC3), catenin (CTNNB1), low density lipoprotein receptor (LDLR), C-reactive protein (CRP), apolipoprotein a (Apo(a)), Factor VII, Factor XI, antithrombin III (SERPINC1), phosphatidylinositol glycan class A (PIG-A), C5, alpha-1 antitrypsin (SERPINA1), hepcidin regulation (TMPRSS6), (delta-aminolevulinate synthase 1 (ALAS-1), acylCaA:diacylglycerol acyltransferase (DGAT), miR-122, miR-21, miR-155, miR-34a, prekallikrein (KLKB1), connective tissue growth factor (CCN2), intercellular adhesion molecule 1 (ICAM-1), glucagon receptor (GCGR), glucocorticoid receptor (GCCR), protein tyrosine phosphatase (PTP-1B), c-Raf kinase (RAF1), fibroblast growth factor receptor 4 (FGFR4), vascular adhesion molecule-1 (VCAM-1), very late antigen-4 (VLA-4), transthyretin (TTR), survival motor neuron 2 (SMN2), growth hormone receptor (GHR), dystrophia myotonic protein kinase (DMPK), cellular nucleic acid-binding protein (CNBP or ZNF9), clusterin (CLU), eukaryotic translation initiation factor 4E (elF-4e), MDM2, MDM4, heat shock protein 27 (HSP 27), signal transduction and activator of transcription 3 protein (STAT3), vascular endothelial growth factor (VEGF), kinesin spindle protein (KIF11), hepatitis B genome, the androgen receptor (AR), Atonal homolog 1 (ATOH1), vascular endothelial growth factor receptor 1 (FLT1), retinoschism 1 (RS1), retinal pigment epithelium-specific 65 kDa protein (RPE65), Rab escort protein 1 (CHM), and the sodium channel, voltage gated, type X, alpha subunit (PN3 orSCN10A). Additional proteins of interest that may be targeted by degron tag insertion include proteins associated with gain of function mutations, for example, cancer causing proteins.

In particular embodiments, the protein of interest is apoB-100, ANGPTL3, PCSK9, APOC3, CRP, ApoA, Factor XI, Factor VII, antithrombin III, phosphatidylinositol glycan class A (PIG-A), the C5 component of complement, Alpha-1-antitrypsin (A1AT), TMPRSS6, ALAS-1, DGAT-2, KLB1, CCN2, ICAM, glucagon receptor, glucocorticoid receptor, PTP-1B, FGFR4, VCAM-1, VLA-4, GCCR, TTR, SMN1, GHR, DMPK, or sodium channel isoform NaV1.8.

In one embodiment, the degron tag is genomically integrated in-frame, either 5′ or 3′, into the gene encoding for an endogenous protein associated with a proteopathy. In one embodiment the degron tag is genomically integrated in-frame, either 5′ or 3′, into the gene encoding for an endogenous protein associated with a disorder such as Alzheimer's disease (Amyloid peptide (Ab); Tau protein), Cerebral b-amyloid angiopathy (Amyloid b peptide (Ab)), Retinal ganglion cell degeneration in glaucoma (Amyloid b peptide (Ab)), Prion diseases (Prion protein), Parkinson's disease and other synucleinopathies (a-Synuclein), Tauopathies (Microtubule-associated protein tau (Tau protein)), Frontotemporal lobar degeneration (FTLD) (Ubi+, Tau) (TDP-43), FTLD-FUS (Fused in sarcoma (FUS) protein), Amyotrophic lateral sclerosis (ALS) (Superoxide dismutase, TDP-43, FUS), Huntington's disease and other triplet repeat disorders (Proteins with tandem glutamine expansions), Familial British dementia (ABri), Familial Danish dementia (Adan), Hereditary cerebral hemorrhage with amyloidosis (Icelandic) (HCHWA-1) (Cystatin C), CADASIL (Notch3), Alexander disease (Glial fibrillary acidic protein (GFAP)), Seipinopathies (Seipin), Familial amyloidotic neuropathy, Senile systemic amyloidosis (Transthyretin), Serpinopathies (Serpins), AL (light chain) amyloidosis (primary systemic amyloidosis) (Monoclonal immunoglobulin light chains), AH (heavy chain) amyloidosis (Immunoglobulin heavy chains), AA (secondary) amyloidosis (Amyloid A protein), Type II diabetes (Islet amyloid polypeptide (IAPP; amylin)), Aortic medial amyloidosis (Medin (lactadherin)), ApoAI amyloidosis (Apolipoprotein AI), ApoAII amyloidosis (Apolipoprotein AII), ApoAIV amyloidosis (Apolipoprotein AIV), Familial amyloidosis of the Finnish type (FAF) (Gelsolin), Lysozyme amyloidosis (Lysozyme), Fibrinogen amyloidosis (Fibrinogen), Dialysis amyloidosis (Beta-2 microglobulin), Inclusion body myositis/myopathy (Amyloid b peptide (Ab)), Cataracts (Crystallins), Retinitis pigmentosa with rhodopsin mutations (rhodopsin), Medullary thyroid carcinoma (Calcitonin), Cardiac atrial amyloidosis (Atrial natriuretic factor), Pituitary prolactinoma (Prolactin), Hereditary lattice corneal dystrophy (Keratoepithelin), Cutaneous lichen amyloidosis (Keratins), Mallory bodies (Keratin intermediate filament proteins), Corneal lactoferrin amyloidosis (Lactoferrin), Pulmonary alveolar proteinosis (Surfactant protein C (SP-C)), Odontogenic (Pindborg) tumor amyloid (Odontogenic ameloblast-associated protein), Seminal vesicle amyloid (Semenogelin I), Cystic Fibrosis (cystic fibrosis transmembrane conductance regulator (CFTR) protein), Sickle cell disease (Hemoglobin), and Critical illness myopathy (CIM) (Hyperproteolytic state of myosin ubiquitination).

In-frame insertion of the nucleic acid sequence encoding the degron tag can be performed or achieved by any known and effective genomic editing processes. In one aspect, the present invention utilizes the clustered regularly interspaced short palindromic repeats (CRISPR)-Cas9 system to produce knock-in endogenous protein-degron tag fusion proteins that are produced from the endogenous locus and are readily degraded in a reversible and dose-responsive fashion dependent on administration of an IMiD or CELMoD. In certain embodiments, the CRISPR-Cas9 system is employed in order to insert an expression cassette for degron tag present in a homologous recombination (HR) “donor” sequence with the degron tag nucleic acid sequence serving as a “donor” sequence inserted into the genomic locus of a protein of interest during homologous recombination following CRISPR-Cas endonucleation. The HR targeting vector contains homology arms at the 5′ and 3′ end of the expression cassette homologous to the genomic DNA surrounding the targeting gene of interest locus. By fusing the nucleic acid sequence encoding the degron tag in frame with the target gene of interest, the resulting fusion protein contains a degron tag that is targeted by a CRBN-IMiD complex or a CRBN-CELMOD complex.

In certain embodiments, a nucleic acid or vector encoding a degron tag or a fusion protein as described herein may be introduced into a cell, whereby the encoded degron tag or fusion protein is expressed by the cell. Accordingly, cells which express a nucleic acid sequence described herein or a vector described herein are also provided. Cells that express a degron or fusion protein of the invention are also provided herein.

The cells may be any suitable cells. In a specific embodiment, the cells are immune effector cells. For example, the cells may selected from the group consisting of: a T cell, B cell, plasma cell, NK cell, NKT cell, innate lymphoid cell, macrophage, dendritic cell, monocyte, neutrophil, basophil, eosinophil, mast cell, hematopoietic progenitor cell, hematopoietic stem cell, other adult stem cell such as neural, cornea, muscle, skin, small intestine, colon, bone, mesenchyme, embryonic stem cell and an induced pluripotent stem cell. The cells may be derived from a mammal, for example, they may be a human cell, or a cell derived from a non-human mammal such as a monkey, a mouse, a rat, a pig, a horse, or a dog.

For example, a cell collected, isolated, purified or induced from a body fluid, a tissue or an organ such as blood (peripheral blood, umbilical cord blood, etc.) or bone marrow can be used. A peripheral blood mononuclear cell (PBMC), an immune cell (a dendritic cell, a B cell, a hematopoietic stem cell, a macrophage, a monocyte, a NK cell or a hematopoietic cell (a neutrophil, a basophil)), an umbilical cord blood mononuclear cell, a fibroblast, a precursor adipocyte, a hepatocyte, a skin keratinocyte, a mesenchymal stem cell, an adipose stem cell, various cancer cell strains, or a neural stem cell can be used. In the present invention, use of a T-cell, a precursor cell of a T-cell (a hematopoietic stem cell, a lymphocyte precursor cell etc.) or a cell population containing them is preferable. Representative examples of T-cells include CD8-positive T-cells, CD4-positive T-cells, regulatory T-cells, cytotoxic T-cells, and tumor infiltrating lymphocytes. The cell population containing a T-cell and a precursor cell of a T-cell includes a PBMC. The aforementioned cells may be collected from a living body, obtained by expansion culture of a cell collected from a living body, or established as a cell strain. When transplantation of the degron- or fusion protein-expressing cell into a living body is desired, it is preferable to introduce the nucleic acid into a cell collected from the living body itself or a conspecific living body thereof. Thus, the immune effector cells may be autologous or allogeneic.

Immune effector cells expressing the degron or fusion protein of the present invention can be engineered by introducing a nucleic acid encoding the degron or fusion protein into a cell. In one embodiment, the step is carried out ex vivo. For example, a cell can be transformed ex vivo with a vector carrying the nucleic acid of the present invention to produce a cell expressing the degron or fusion protein of the present invention.

The immune effector cells expressing the fusion protein containing the POI and the degron tag can be used as a therapeutic agent for a disease. The therapeutic agent can be the cell expressing the POI as an active ingredient, and may further include a suitable excipient. The disease against which the cell expressing the POI is administered is not limited as long as the disease shows sensitivity to the transformed immune effector cells. Representative examples of diseases treatable with immune effector cells expressing nucleic acids encoding fusion proteins containing the POI and a degron tag include cancer (neuroblastoma, blood cancer (leukemia), solid tumor, etc.), inflammatory disease/autoimmune disease (asthma, eczema), hepatitis, and infectious disease, e.g., the cause of which is a virus such as influenza and HIV, a bacterium, or a fungus, for example, tuberculosis, MRSA, VRE, and deep mycosis. The transformed immune effector cells can bind to an antigen presented by a target cell that is desired to be decreased or eliminated for treatment of the aforementioned diseases, that is, a tumor antigen, a viral antigen, a bacterial antigen or the like is administered for treatment of these diseases.

The immune effector cells may be administered intradermally, intramuscularly, subcutaneously, intraperitoneally, intranasally, intraarterially, intravenously, intratumorally, or into an afferent lymph vessel, by parenteral administration, for example, by injection or infusion, although the administration route is not limited. The cells may be injected, for example, directly into a tumor, lymph node, or site of infection.

The degron tags provided herein bind to CRBN-IMiD complexes, or CRBN-CELMOD complexes. When the degrons provided herein are comprised within a fusion protein (e.g. a degron-POI fusion protein, such as a degron-CAR fusion protein), they can bind to CRBN-IMiD complexes, or CRBN-CELMOD complexes, and induce degradation of the fusion protein in a cell. The degrons can therefore advantageously be used as a “safety switch” by inducing degradation of a POI in situations where expression of the POI is undesirable.

It is noted that some IMiDs, for example pomolidomide, are efficient at crossing the blood-brain barrier. Hence modulation of iTAG2 would be highly relevant for brain tumours in adults and children amenable to treatment with CAR-T cells, for example high grade glioma, medulloblastoma meningioma.

IMiD (immunomodulatory drugs) and CELMoD (cereblon modulators) compounds are known in the art, examples of which include thalidomide, pomalidomide, lenalidomide, CC-122, CC-220 and CC-885, or pharmaceutically acceptable salts thereof (e.g., HCl salt). The IMiD compounds, thalidomide (marketed under the name THALOMID®), lenalidomide (marketed under the name REVLIMID®) and pomalidomide (marketed under the name POMALYST®), have each been approved by the FDA for treatment of multiple myeloma (among other diseases). THALOMID® is currently available as capsules containing 50 mg, 100 mg, 150 mg or 200 mg thalidomide. REVLIMID® is currently available as capsules containing 2.5 mg, 5 mg, 10 mg, 15 mg, 20 mg or 25 mg lenalidomide. POMALYST® is currently available as capsules containing 1 mg, 2 mg, 3 mg or 4 mg pomalidomide. The CELMoD compounds CC-122, CC-220 and CC-885 are currently undergoing review by the FDA.

In a particular example, degradation of the degron tags or fusion proteins provided herein is lenalidomide-dependent. In another example, degradation of the degron tags or fusion proteins provided herein is pomalidomide-dependent. In a further example, degradation of the degron tags or fusion proteins provided herein is iberdomide-dependent. As would be known to a person of skill in the art, iberdomide is also referred to as CC-220, and these terms are used interchangeably herein. Similarly, the terms “avadomide” and “CC-122” are used interchangeably herein.

IMiD and CELMoD compounds may be in the form of a free acid or free base, or a pharmaceutically acceptable salt. As used herein, the term “pharmaceutically acceptable” in the context of a salt refers to a salt of the compound that does not abrogate the biological activity or properties of the compound, and is relatively non-toxic, i.e., the compound in salt form may be administered to a subject without causing undesirable biological effects (such as dizziness or gastric upset) or interacting in a deleterious manner with any of the other components of the composition in which it is contained. The term “pharmaceutically acceptable salt” refers to a product obtained by reaction of the compound of the present invention with a suitable acid or a base. Examples of pharmaceutically acceptable salts of the IMiD and CELMoD compounds include those derived from suitable inorganic bases such as Li, Na, K, Ca, Mg, Fe, Cu, Al, Zn and Mn salts. Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or β-toluenesulfonate salts and the like. Certain compounds of the invention can form pharmaceutically acceptable salts with various organic bases such as lysine, arginine, guanidine, diethanolamine or metformin.

IMiD and CELMoD compounds may have at least one chiral centre and thus may be in the form of a stereoisomer, which as used herein, embraces all isomers of individual compounds that differ only in the orientation of their atoms in space. The term stereoisomer includes mirror image isomers (enantiomers which include the (R-) or (S-) configurations of the compounds), mixtures of mirror image isomers (physical mixtures of the enantiomers, and racemates or racemic mixtures) of compounds, geometric (cis/trans or E/Z, R/S) isomers of compounds and isomers of compounds with more than one chiral centre that are not mirror images of one another (diastereoisomers). The chiral centres of the compounds may undergo epimerization in vivo; thus, for these compounds, administration of the compound in its (R-) form is considered equivalent to administration of the compound in its (S-) form. Accordingly, the IMiD and CELMoD compounds may be used in the form of individual isomers and substantially free of other isomers, or in the form of a mixture of various isomers, e.g., racemic mixtures of stereoisomers.

In some embodiments, the IMiD or CELMoD compound is an isotopic derivative in that it has at least one desired isotopic substitution of an atom, at an amount above the natural abundance of the isotope, i.e., enriched. In one embodiment, the compound includes deuterium or multiple deuterium atoms. Substitution with heavier isotopes such as deuterium, i.e. ²H, may afford certain therapeutic advantages resulting from greater metabolic stability, for example, increased in vivo half-life or reduced dosage requirements, and thus may be advantageous in some circumstances.

In addition, IMiD and CELMoD compounds embrace the use of N-oxides, crystalline forms (also known as polymorphs), active metabolites of the compounds having the same type of activity, tautomers, and unsolvated as well as solvated forms with pharmaceutically acceptable solvents such as water, ethanol, and the like, of the compounds. The solvated forms of the conjugates presented herein are also considered to be disclosed herein.

A pharmaceutical composition comprising a degron tag, fusion protein, nucleotide sequence, vector, or cell of the invention, and a pharmaceutically acceptable excipient, carrier, adjuvant and/or diluent is also provided herein. Pharmaceutical compositions may routinely contain pharmaceutically acceptable concentrations of salt, buffering agents, preservatives, compatible carriers, supplementary immune potentiating agents such as adjuvants and cytokines and optionally other therapeutic agents or compounds.

As used herein, “pharmaceutically acceptable” refers to a material that is not biologically or otherwise undesirable, i.e., the material may be administered to an individual along with the selected compound without causing any undesirable biological effects or interacting in a deleterious manner with any of the other components of the pharmaceutical composition in which it is contained.

Excipients are natural or synthetic substances formulated alongside an active ingredient (e.g. a compound of the invention), included for the purpose of bulking-up the composition or to confer a therapeutic enhancement on the active ingredient in the final dosage form, such as facilitating drug absorption or solubility. Excipients can also be useful in the manufacturing process, to aid in the handling of the active substance concerned such as by facilitating powder flowability or non-stick properties, in addition to aiding in vitro stability such as prevention of denaturation over the expected shelf life. Pharmaceutically acceptable excipients are well known in the art. A suitable excipient is therefore easily identifiable by one of ordinary skill in the art. By way of example, suitable pharmaceutically acceptable excipients include water, saline, aqueous dextrose, glycerol, ethanol, and the like. Adjuvants are pharmacological and/or immunological agents that modify the effect of other agents in a composition. Pharmaceutically acceptable adjuvants are well known in the art. A suitable adjuvant is therefore easily identifiable by one of ordinary skill in the art. Diluents are diluting agents. Pharmaceutically acceptable diluents are well known in the art. A suitable diluent is therefore easily identifiable by one of ordinary skill in the art. Carriers are non-toxic to recipients at the dosages and concentrations employed and are compatible with other ingredients of the formulation. The term “carrier” denotes an organic or inorganic ingredient, natural or synthetic, with which the active ingredient is combined to facilitate the application. Pharmaceutically acceptable carriers are well known in the art. A suitable carrier is therefore easily identifiable by one of ordinary skill in the art.

The pharmaceutical compositions of the invention may be used as a medicament. As detailed above they may be particularly useful for immune cell therapy, wherein the degron tags provided herein may act as a safety switch to modulate the expression of a POI.

A method of degrading a protein of interest (POI) is therefore also provided, comprising: contacting a cell in vitro or in vivo with an effective amount of an immunomodulatory drug (IMiD) or a cereblon modulator (CELMoD), or a pharmaceutically acceptable salt or stereoisomer thereof, wherein the cell expresses a nucleic acid encoding a fusion protein of the invention (i.e. a fusion protein comprising a POI and a degron tag of the invention). The methods may be conducted in vivo or in vitro. The POIs may be exogenous or endogenous.

The cells may be allogeneic or autologous. The method may be performed on a subject, in vivo. The subject may be human.

A method of degrading a protein of interest is also provided, comprising:

administering an effective amount of an immunomodulatory drug (IMiD) or a cereblon modulator (CELMoD), or a pharmaceutically acceptable salt or stereoisomer thereof, to a subject, wherein the subject has previously been treated via gene therapy causing at least some endogenous cells to express a nucleic acid encoding a fusion protein of the invention (i.e. a fusion protein comprising a POI and a degron tag of the invention). Gene therapy is a medical approach that treats or prevents a disease by correcting the underlying genetic problem via the introduction of genes into cells. The gene therapy comprise introducing a nucleic acid or vector of the invention into the subject, wherein the nucleic acid or vector encodes a degron tag or fusion protein described herein (i.e. a fusion protein comprising a POI and a degron tag of the invention). The nucleic acid or vector may be introduced into cells ex vivo, that are then introduced into the subject, or the nucleic acid or vector may be introduced into cells of the subject in vivo. In some embodiments, the subject has been administered immune effector cells such as autologous T-cells (CAR-T cells) which have been genetically modified to express a chimeric antigen receptor protein (CAR)-degron tag fusion protein, and is experiencing an adverse immune response (e.g., cytokine release syndrome or neurotoxicity) as a result of the therapy. In some other embodiments, the gene therapy includes gene knock-in, administration of viral vectors or clustered regularly interspaced short palindromic repeats (CRISPR)-mediated knock in.

Yet a further aspect of the invention is directed to a method of reducing gene overexpression in a subject including introducing into one or more relevant cells of the subject a nucleic acid sequence encoding a degron tag that is integrated genomically in-frame with a nucleic acid sequence of an endogenous protein associated with a disease due to overexpression of the endogenous protein; and administering to the subject an effective amount of an IMiD or CELMoD. In some embodiments, the endogenous protein is associated with a disease that is a result of a gain of function mutation, amplification or increased expression, a monogenetic disease, a proteopathy, or a combination thereof.

A further aspect of the invention is directed to a method of evaluating the function of an endogenous protein or validating an endogenous protein as a target for therapy of a disease state including introducing into one or more relevant cells a nucleic acid sequence encoding a degron tag that is integrated genomically in-frame with a nucleic acid sequence of an endogenous protein suspected of being associated with a disease; and contacting the cells with an effective amount of an IMiD or CELMoD. The methods may be conducted in vivo (e.g. in animal models) or in vitro (e.g. in cell cultures).

Any of the inventive methods may entail contacting the cell or administering to the subject an IMiD or CELMoD which is thalidomide, pomalidomide, lenalidomide, CC-122, CC-220 or CC-885.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

Aspects of the invention are demonstrated by the following non-limiting examples.

### Examples

Thalidomide-like derivatives (called immunomodulatory drugs IMiDs, or more recently, Cereblon E3 ligase modulators CELMoDs) are degrader drugs that bind to a conserved tri-tryptophan cage on the surface of Cereblon (CRBN), the substrate receptor of the Cullin4-RING E3 ubiquitin ligase CRL4^{CRBN}, and induce the alteration of CRL4^{CRBN} substrate specificity, leading to the recruitment, ubiquitination and subsequent proteasomal degradation of neo-substrates such as Ikaros (IKZF1), Aiolos (IKZF3), Casein Kinase 1 alpha (CK1a or CSNK1A), G₁ to S phase transition protein 1 (GSPT1) and zinc finger protein 91 (ZFP91) (Table 1) 7,11-16

TABLE 1
Summary of IMiDs/CELMoDs Substrate specificity: Data from Thoma and co-
workers ⁸, Ebert/Fischer and co-workers ^{7,17,18}, Chamberlain and co-workers ¹⁶

| | | | | | Non zinc finger | |
|---|---|---|---|---|---|---|
| | | C2H2 zinc finger | | | containing | |
| | | containing proteins | | | proteins | |
| | | degraded | | | degraded | |
| | | Ikaros | Aiolos | | | Casein |
| Degrader | 2D structure | (IKZF1) | (IKZF3) | ZFP91 | GSPT1 | Kinase 1a |
| Thalidomide | 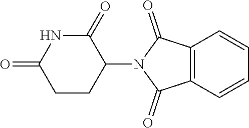 [SMILES: O=C1CCC(N2C(=O)c3ccccc3C2=O)C(=O)N1] | X | X | | | |
| Lenalidomide | 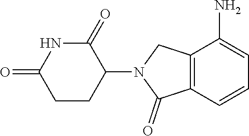 [SMILES: Nc1cccc2c1CN(C1CCC(=O)NC1=O)C2=O] | X | X | | | X |
| Pomalidomide | 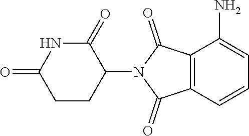 [SMILES: Nc1cccc2c1C(=O)N(C1CCC(=O)NC1=O)C2=O] | X | X | X | | |
| Iberdomide/ CC220 | 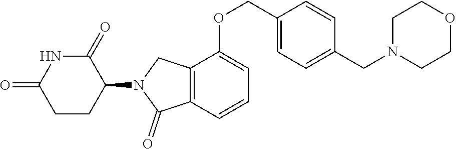 [SMILES: O=C1CC[C@H](N2Cc3c(OCc4ccc(CN5CCOCC5)cc4)cccc3C2=O)C(=O)N1] | X | X | X | | |
| Avadomide/ CC122 | 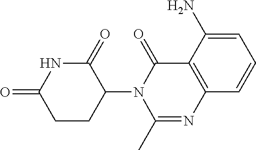 [SMILES: Cc1nc2cccc(N)c2c(=O)n1C1CCC(=O)NC1=O] | X | X | X | | |
| CC885 | 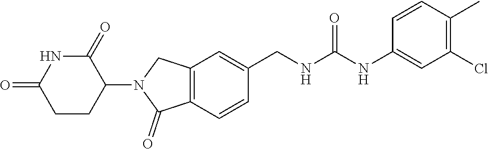 [SMILES: Cc1ccc(NC(=O)NCc2ccc3c(c2)CN(C2CCC(=O)NC2=O)C3=O)cc1Cl] | X | X | | X | |

Neo-substrates interact with the degrader-bound surface of CRBN through recognition motifs known as degrons, characterised by a common β-hairpin loop with a conserved glycine at the apex, which is crucial for the interaction of the degron with the degrader compound. The amino acid sequences adjacent to the sentinel glycine in the β-hairpin loop are variable among neo-substrates and are critical for the specificity of recruitment and degradation of neo-substrates by a particular CRBN binding agent. In the case of the neo-substrates Ikaros, Aiolos and ZFP91, the degron motif is located within a C21H2 zinc finger (ZF) domain, with the first half of the domain harbouring a CxxCG sequence signature containing the conserved glycine just after the second Cysteine of the ZF^{7,8}.

### Generating DCD-EGFP Fusion Constructs

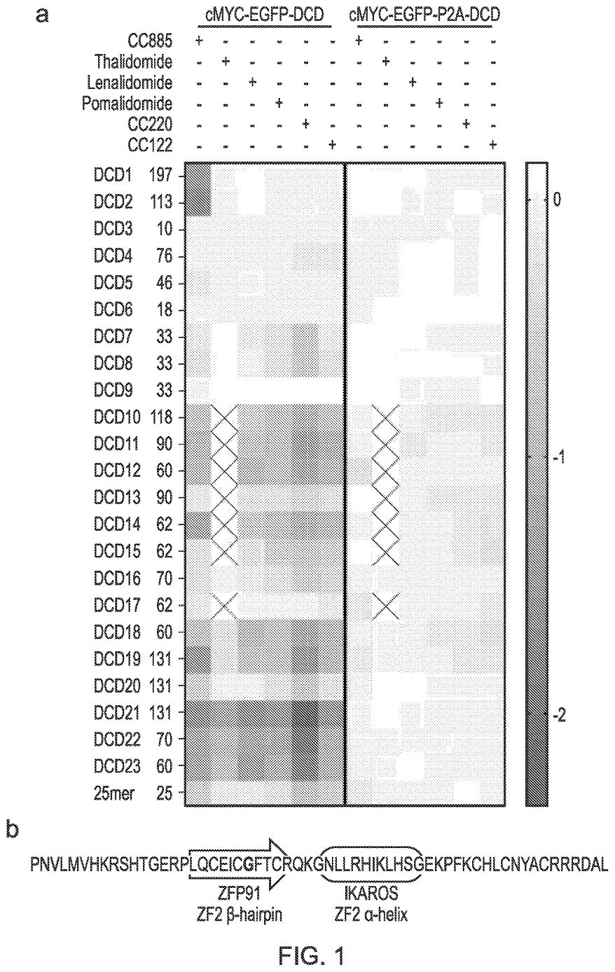

To address the limitations of the currently available protein modulation tools, the inventors developed a novel and robust system for targeted protein degradation by exploiting the fusion of an IMiD/CeLMOD recognition sequence to a protein of interest. By systematically evaluating a matrix of 23 degron containing domains (DCDs) from various ZF and non-ZF proteins with a panel of IMiD/CeLMOD small molecules (see FIG. 1), they identified a DCD tag (DCD23) based on a chimeric sequence of Ikaros and ZFP91, that induces acute target degradation in vitro and in vivo (FIG. 1). This 60 amino acid sequence is referred to as iTAG1 (DCD23) herein.

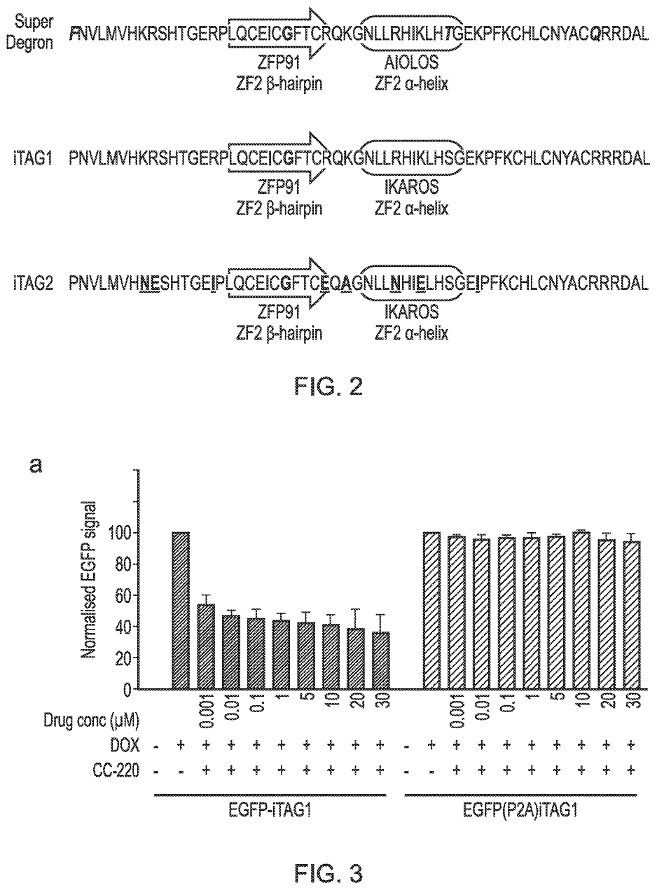

Based on structural analysis of the DCD23 sequence, mutations were introduced to assess whether an improved degron sequence could be identified. A number of different constructs were tested (see e.g. iTAG2v1, iTAG2v2, iTAG2) and an optimal construct was chosen, which is referred to as DCD23mut or iTAG2 herein (FIG. 2).

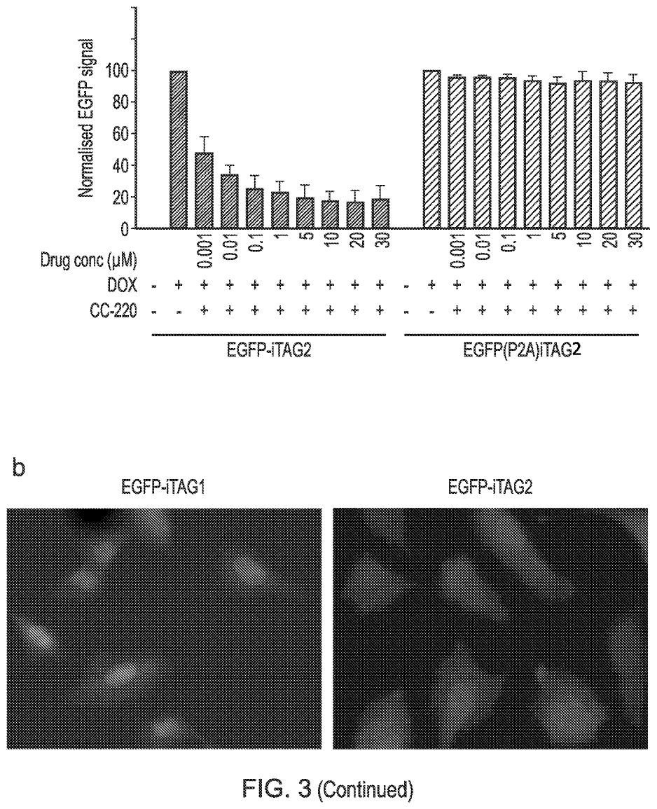

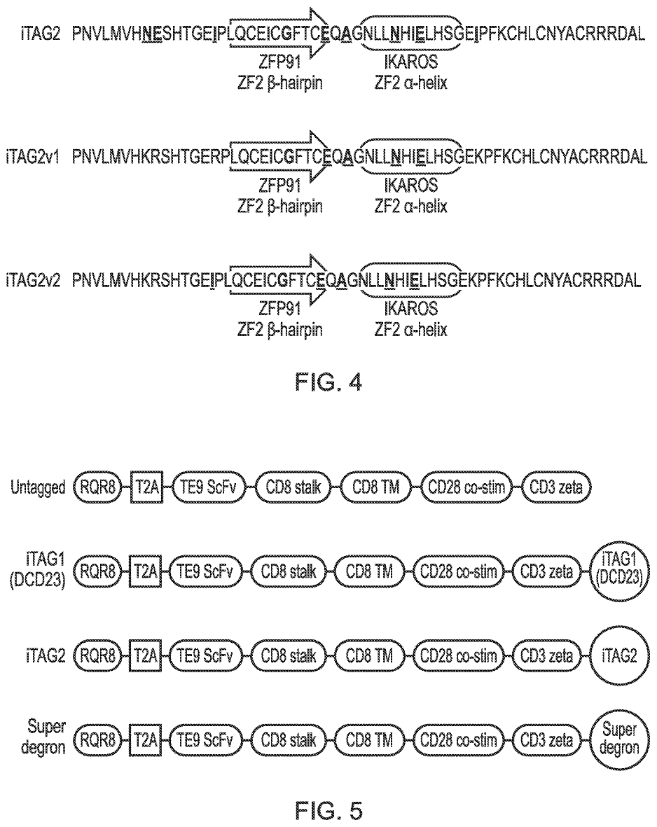

The inventors saw that iTAG2 induced similar degradation to iTAG1(DCD23) when fused to an EGFP construct (FIG. 3.a) and abolished nuclear accumulation (FIG. 3.b). Variations of iTAG2 also contemplated herein are shown in FIG. 4.

Generating iTAG2-Chimeric Antigen Receptor (CAR) Fusion Constructs

The inventors then cloned iTAG2 and iTAG1(DCD23) respectively to the C terminus of a second generation anti-B7H3 chimeric antigen receptor containing CD28 and CD3zeta endodomains. The tagged CARs were co-expressed from a gammaretroviral vector via 2A sequence with a CD34 marker gene (FIG. 5). The inventors sought to determine if the two tagged CARs were successfully degraded following exposure of cells expressing the tagged CARs to IMiD drugs.

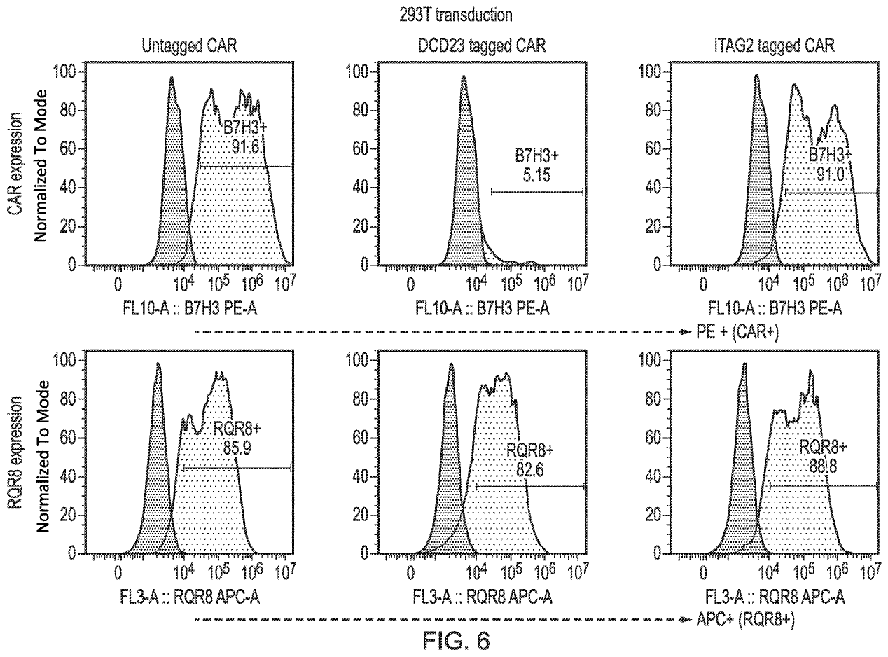

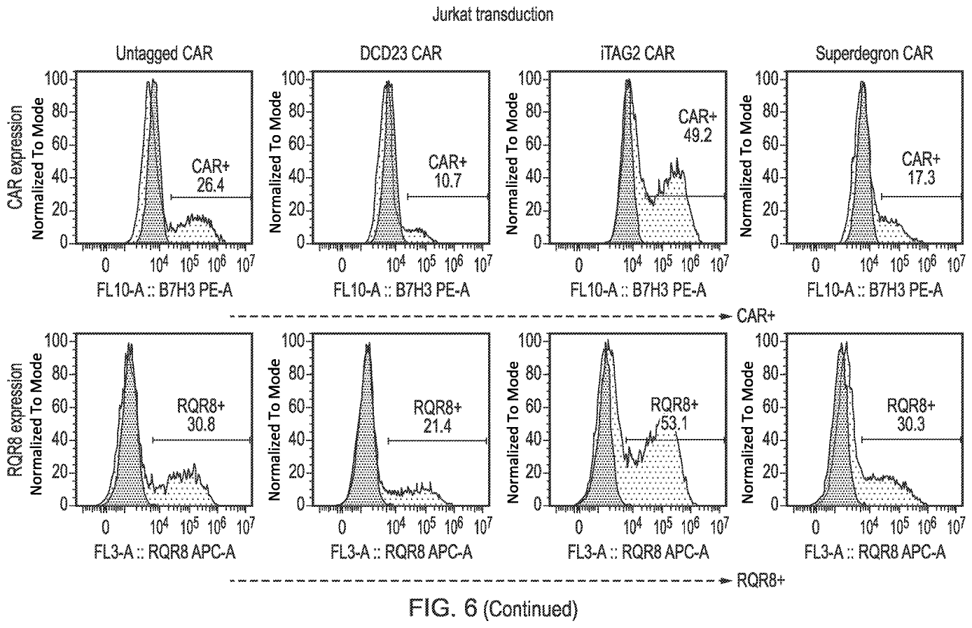

Initial experiments evaluated expression and degradation in human Jurat T cell leukaemia cells or human 293T cells, which were stably transduced with untagged, iTAG1(DCD23)-tagged, and iTAG2-tagged anti-B7H3 CAR-T cells. Here the relative expression of the CAR and the CD34 marker gene are determined by direct staining and evaluation by flow cytometry. In both target cell lines, whilst the iTAG2-CAR and the untagged CAR have similar levels of expression of CD34 and CAR, for iTAG1(DCD23)-tagged CAR, CAR expression was lower than CD34, indicating relatively poor cell surface expression (FIG. 6). These data show that iTAG2 sequence led to superior cell surface expression of chimeric antigen receptor compared with the iTAG1(DCD23) sequence.

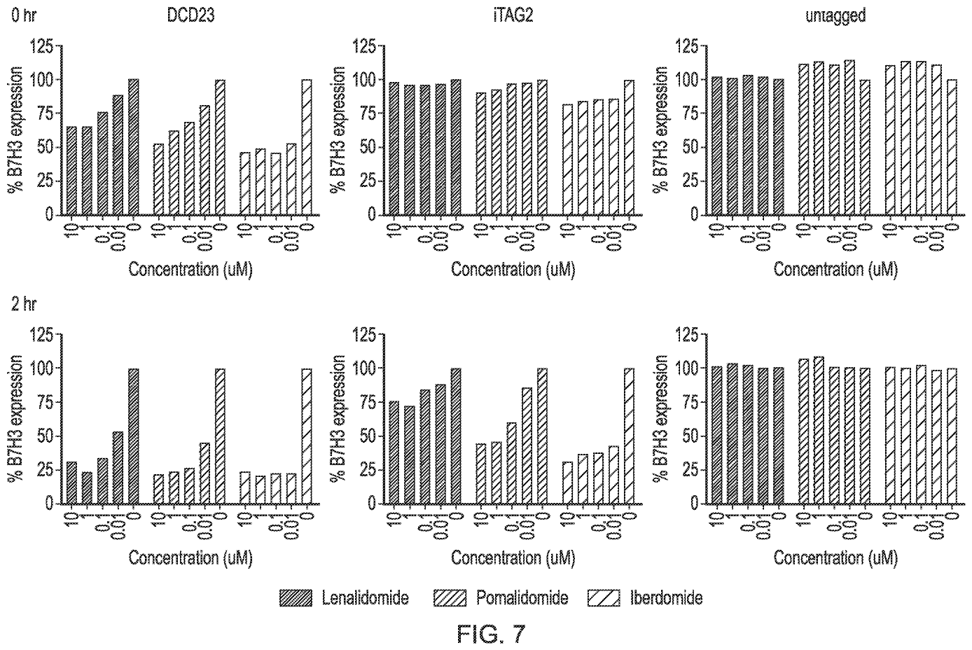

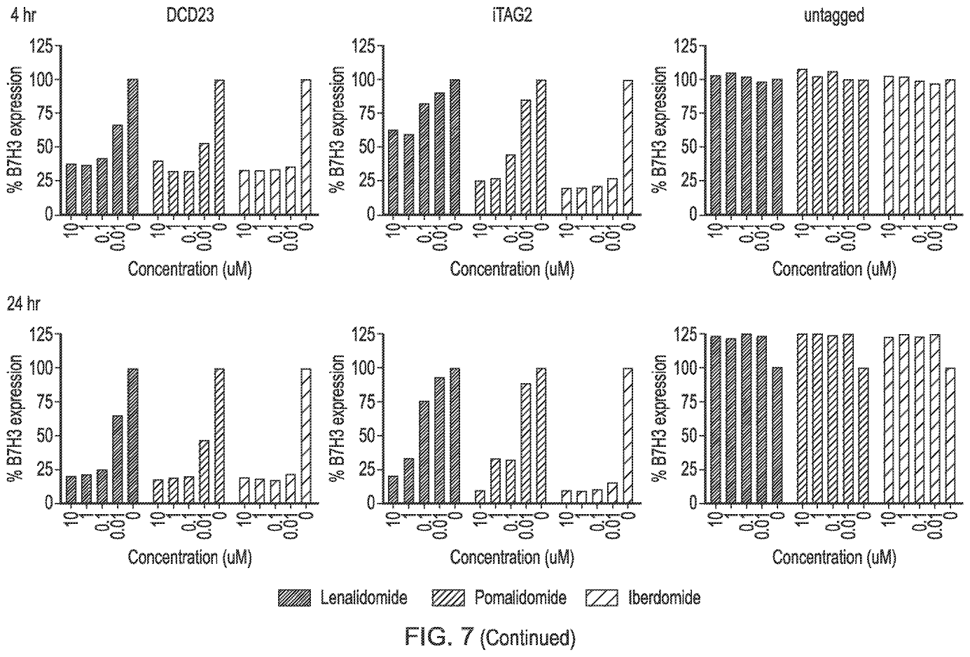

The inventors next evaluated downregulation of iTAG2-tagged CAR in transduced Jurkat cells following addition of a range of concentrations of the IMiD drugs (lenalidomide, pomalidomide and iberdomide). Both the iTAG1(DCD23) tagged and iTAG2 tagged CARs showed similar patterns of downregulation following addition of drug, the maximal reduction of surface expression being detectable after 4 hours of drug treatment. Iberdomide showed the highest potency for degradation with nearly maximal downregulation being observed at as little as 0.01 micromolar drug concentration (FIG. 7).

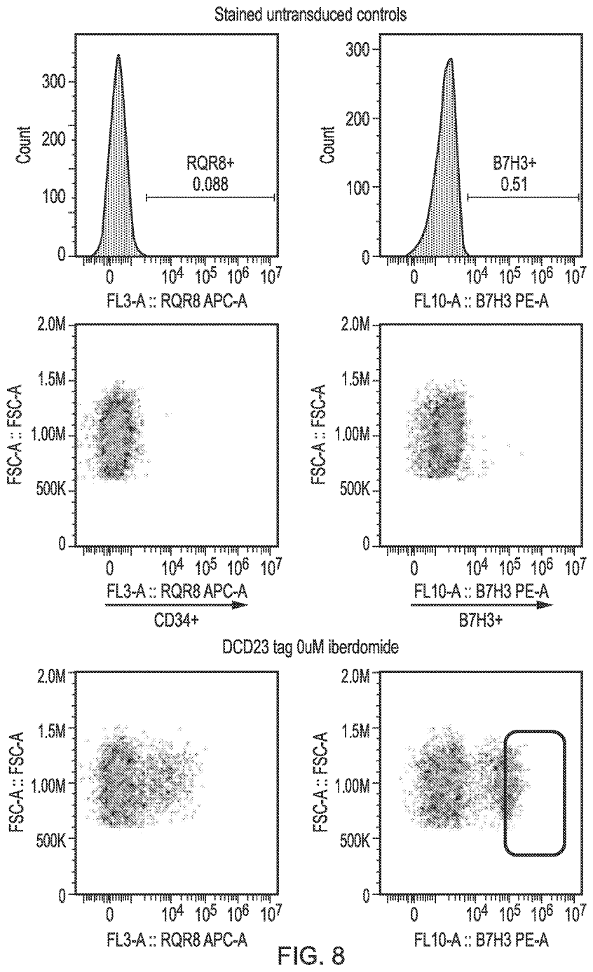

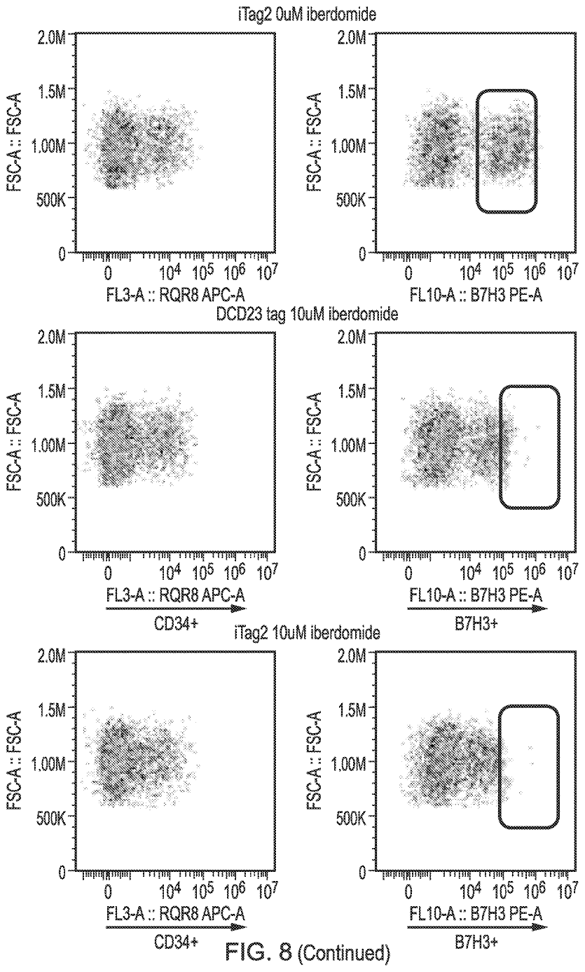

Having demonstrated expression and drug-induced degradation in the Jurkat cell line, the inventors then evaluated these parameters in primary human T cells. Human PBMC from two independent donors were transduced with the TE9-28Z anti-B7H3 CAR in the untagged, DCD23 and iTAG2 formats, and expanded for 7 days before evaluation of CAR expression following addition of 10 uM iberdomide for 24 hours. Of interest in primary T cells at 24 hours, the iberdomide treatment led to near complete loss of expression of the brightly expressed CAR population although dim expression was still observed (FIG. 8).

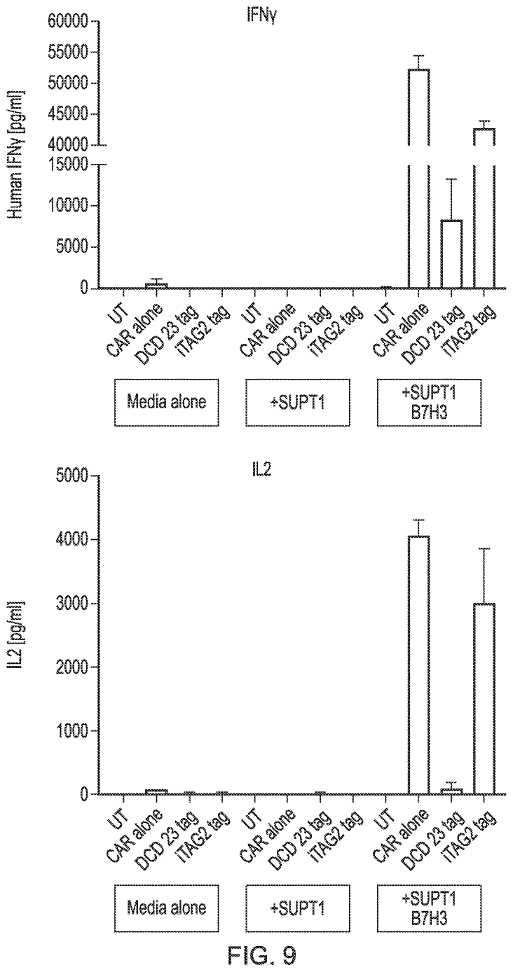

To evaluate whether the brighter CAR expression of iTAG2 tagged CAR compared with DCD23-tagged CAR translated into greater effector function, the respective CAR-T populations from the two independent donors were co-cultured overnight with human SupT1 leukaemia target cells isogenic for the target antigen B7H3. Wild type SupT1 have no detectable B7H3 expression. Following overnight culture of T cells with the targets, culture supernatant was harvested and evaluated for inflammatory cytokines interferon gamma and Interleukin-2 by ELISA (FIG. 9). The data indicate that the DCD23 fusion protein has approximately 5-fold reduced effector function compared with iTAG2 fusion protein in terms of interferon gamma secretion and approximately 30-fold reduced IL-2 secretion (FIG. 9). Effector function of iTGA2 tagged CAR is not significantly different to untagged CAR.

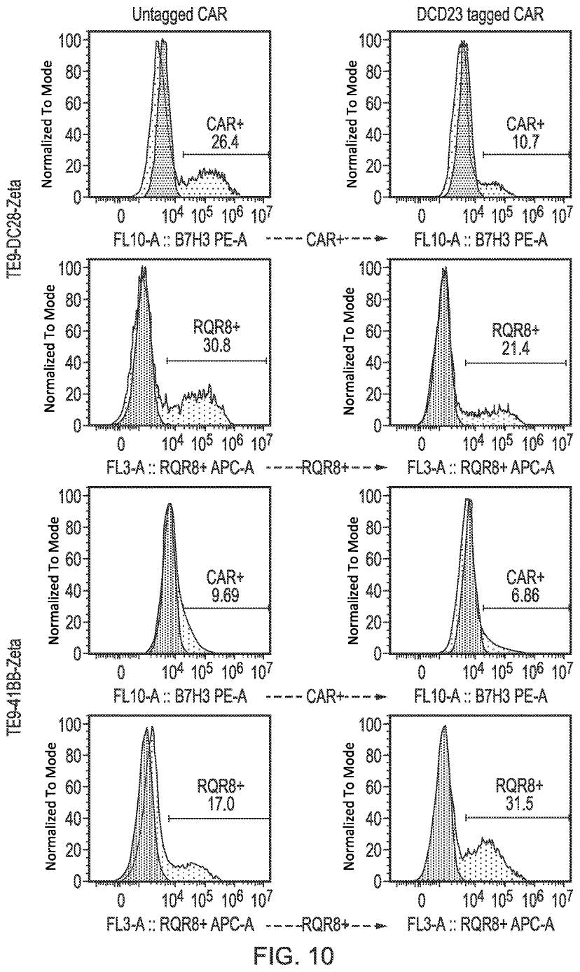

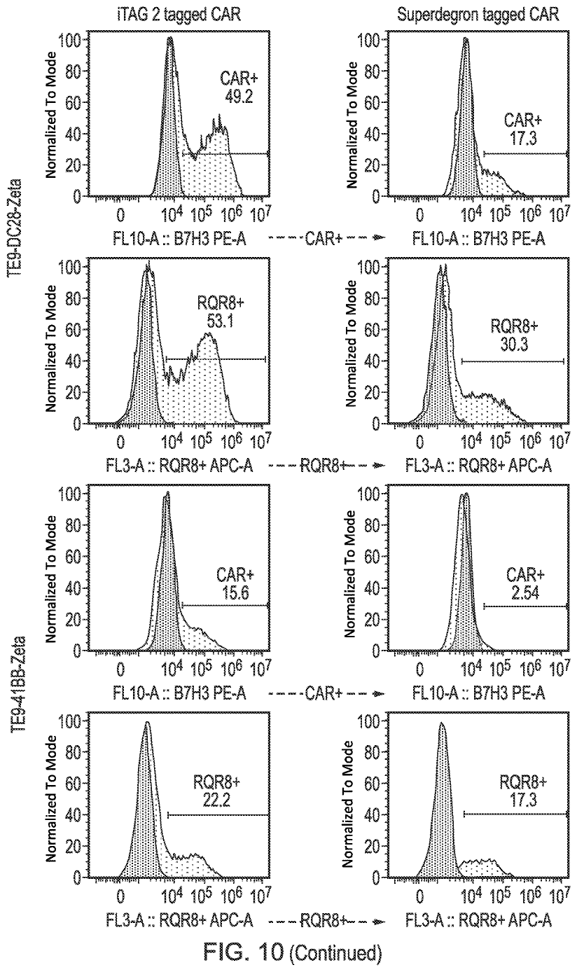

The inventors compared surface expression of both 41BB and CD28 versions of the TE9 CAR and compared surface expression of the iTAG2 fusion protein with both DCD23 and the previously published super-degrader (referred to as SuperDegron herein). In transduced Jurkat cells, there is similar weak surface expression of the DCD23 and SuperDegron tagged CARs whilst in this experiment, the iTAG2 tagged CARs have the same expression as wild type (untagged CARs). Contrary to published report that the SuperDegron worked only in the context of 41 BB CAR, the inventors saw no evidence of differential surface expression between the two costimulatory arrangements (FIG. 10). The data shown herein therefore indicates that the tested DCD sequences (iTAG2, DCD23 and super-degrader) function in both 41BB and CD28 versions of second generation CARs.

Aiolos Peptide-Based TR-FRET Assay for the Measurement of the Relative Affinities of iTAG1 And iTAG2 for the Complexes Formed Between CRBN/DDB1 (DNA Damage Binding Protein 1) and Various IMiDs

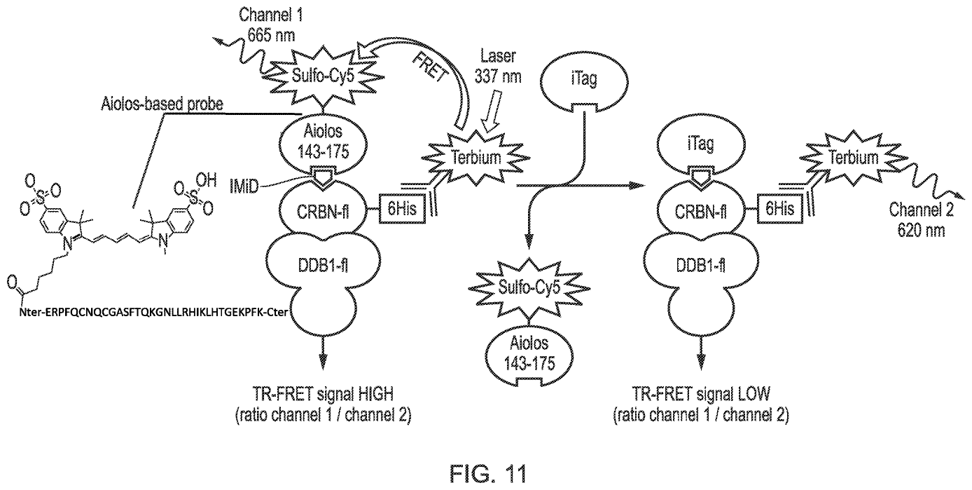

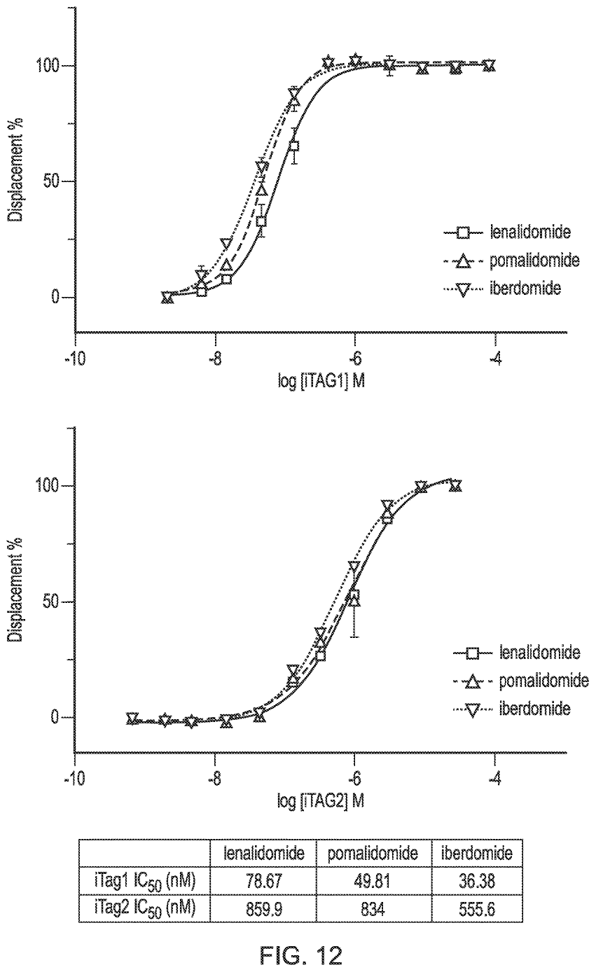

A schematic representation of the Aiolos peptide-based time resolved-Förster resonance energy transfer (TR-FRET) assay is presented in FIG. 11. The inventors used this assay to assess the ability of iTAG1 and iTAG2 to displace a fluorescently labelled Aiolos peptide probe from the complexes formed between CRBN, DDB1 and various IMiDs (lenalidomide, pomalidomide and iberdomide), thereby enabling to measure the relative affinities of iTAG1 and iTAG2 for these complexes. The results are presented in FIG. 12, including the IC₅₀s calculated from the TR-FRET curves. Overall, the IC₅₀s obtained for iTAG2 were higher than those obtained for iTAG1 with about a 10-fold difference. The relative affinities obtained for the 3 compounds followed the same trend with iTAG1 and iTAG2, with the tightest binding obtained with iberdomide, followed by pomalidomide and lenalidomide.

### Superdegron Tagged GFP has Predominantly Nuclear Localisation

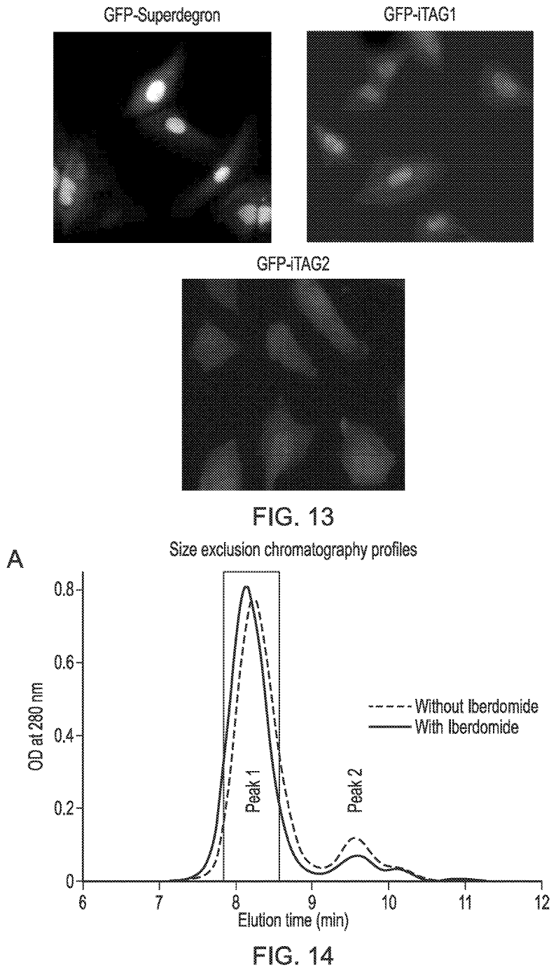

iTAG2 tagged GFP has cytoplasmic localisation in contrast to iTAG1, which has predominant nuclear localisation. To determine localisation of “superdegron” which has a degree of homology with iTAG1, the inventors cloned superdegron into the expression vector to replace iTAG1 as the degron fused with GFP. Following transient transfection in HMEC (Human Mammary Epithelial Cells), the superdegron tag was shown to result in strong nuclear localisation of GFP, shown in FIG. 13.

Size Exclusion Chromatography Confirmation of the Formation of a Complex Between CRBN/DDB1 and iTAG2 in the Presence of Iberdomide

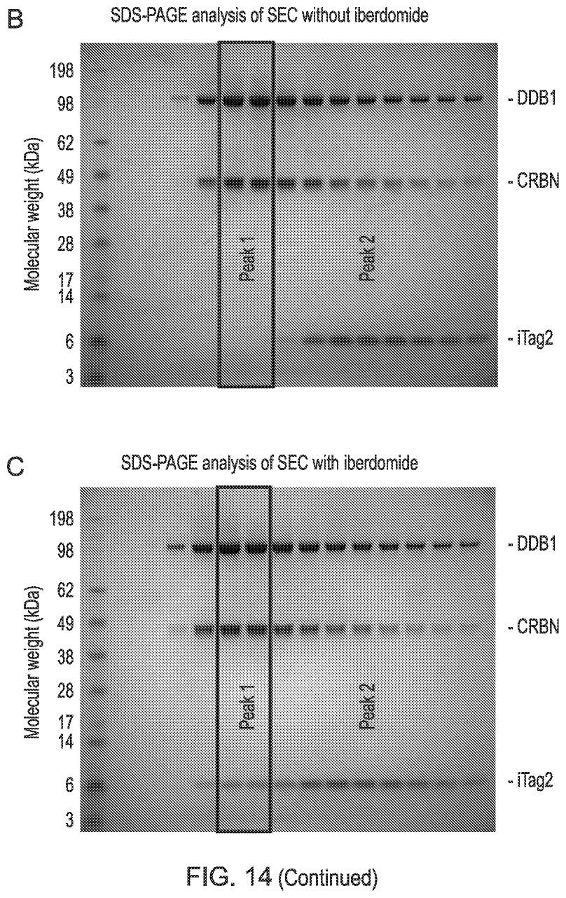

Biochemical evidence for IMiD-dependent formation of a direct interaction between iTAG2 and the CRBN/DDB1 complex was generated using size exclusion chromatography (SEC). Purified CRBN/DDB1 protein complex and iTAG2 protein were mixed together and subjected to SEC in the presence or absence of iberdomide, shown in FIG. 14. The SEC peak 1 corresponding to the higher molecular weight species was shifted towards a lower elution time in the presence of iberdomide, with a slight increase in the optical density at 280 nm, which is consistent with the formation of a larger complex in the presence of iberdomide (FIG. 14A). SDS-PAGE analysis of representative fractions of the SEC analysis in the absence of iberdomide confirmed that the SEC peak 1 contains only DDB1 and CRBN proteins, with iTAG2 only present in the SEC peak 2 (FIG. 14B). In the presence of iberdomide, the SEC peak 1 contains all three proteins, confirming the formation of a complex between DDB1, CRBN and iTAG2 in the presence of iberdomide (FIG. 14C).

iTAG2 Tagged Human CAR-T Cells are Rapidly Re-Expressed Following Cell Washing and Withdrawal of IMiD Drug

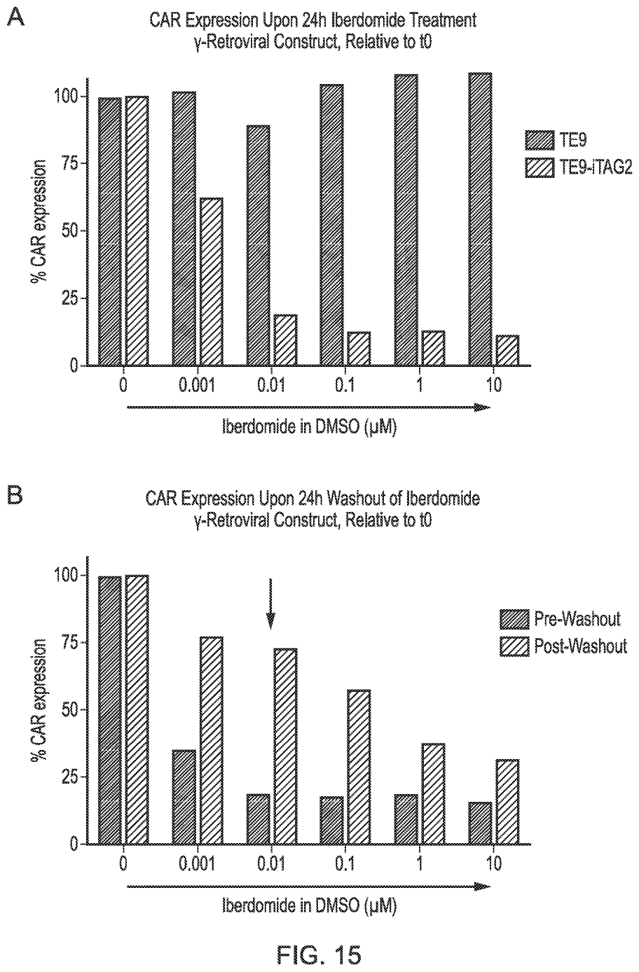

The re-expression of degraded chimeric antigen receptors following removal of the degrader drug is important for the CAR-T cell to be able to toggle between active and resting states, thereby allowing additional therapeutic manipulation and fine-tuning. To determine the reversibility, TE9-28Z-iTAG2 CAR-T cells from human PBMC were treated with iberdomide concentrations to decrease CAR-T surface expression, then washed twice before culturing in fresh media lacking iberdomide for 24 hours to demonstrate re-expression of cell surface CAR, shown in FIGS. 15 A and B.

Anti B7H3 CAR Tagged with iTAG2: Expression and Function in Lentiviral Vector

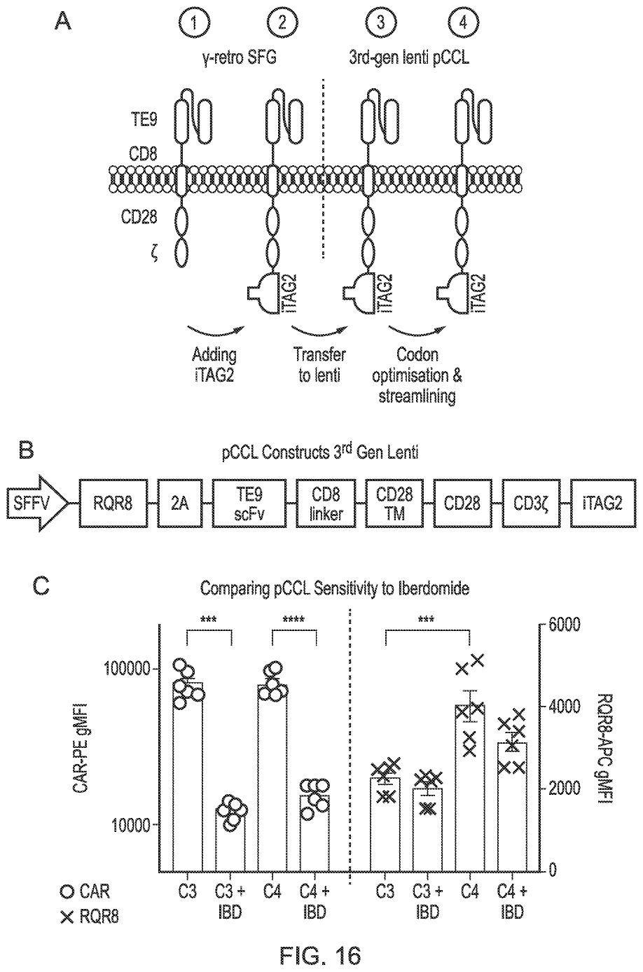

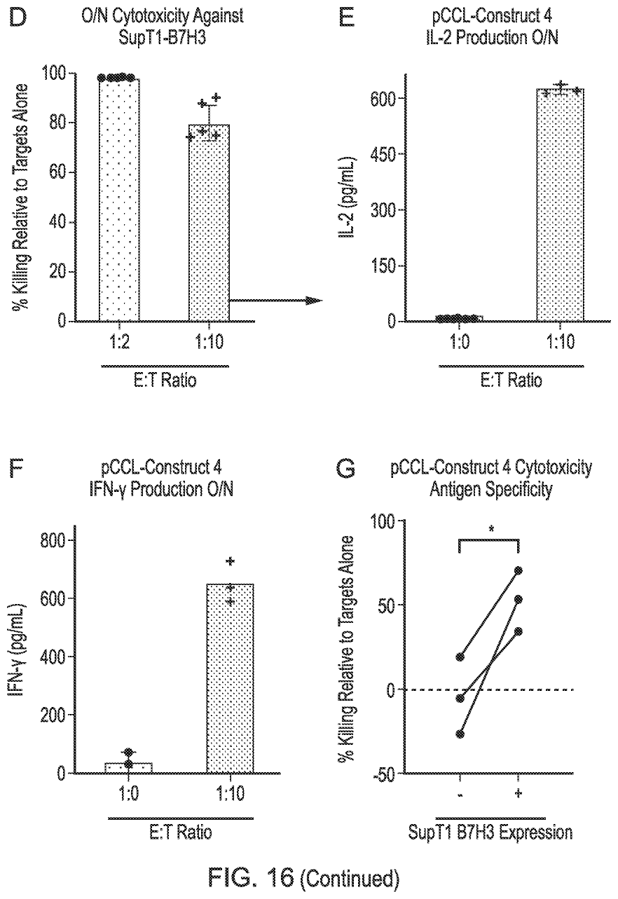

To evaluate the general functionality of the TE9-28Z-iTAG2 anti-B7H3 CAR-T construct when expressed from a different viral vector, driven by alternate promoter, the construct was cloned into third generation lentivector pCLL2 backbone and viral supernatant was generated by transient transfection of 293T cells, with virus pseudotyped with VSV-G. Two versions of the TE9-28Z-iTAG2 sequence were evaluated in this format, which were with and without codon optimisation and removal of cloning scars. There are no differences in amino acid sequence of iTAG2 between original (construct 3) and codon optimised (construct 4) versions (FIG. 16 a and b). To test degradation following codon-optimisation of CAR and iTAG2 sequences, CAR-T cells generated from three independent donors using GMP-like conditions with pCCL2 lentiviral vector transduction, were expanded to 9 days post stimulation and then subjected to overnight treatment with 100 nM iberdomide prior to direct staining of both CAR and co-expressed RQR8 marker gene by flow cytometry. The log reduction in CAR expression as expressed as geometric median fluorescence intensity (gMFI) was highly consistent. The codon optimised iTAG2 in lentiviral backbone was found to show the same degree of sensitivity to iberdomide-induced degradation as the non-codon optimised iTAG2 (FIG. 16c). Codon optimised lentiviral expressed TE9-28z-iTAG2 CAR-T cells show high level of cytotoxicity against antigen-positive target cells as well as inflammatory cytokine secretion (FIG. 16d-f). Lack of codon-optimized TE9-iTAG2 cytotoxicity against B7H3-negative SupT1 targets was confirmed using an overnight luminescence-based assay at a 1:2 E:T ratio (data shown in FIG. 16G for three independent CAR-T donors).

### Methods:

Design of iTAG2

The inventors generated three mutated versions of iTAG1:
iTAG2v1, where only the central ZFP91/lkaros chimeric Zinc-finger were mutated:

iTAG2v1
(SEQ ID NO: 27)
PNVLMVHKRSHTGERPLQCEICGFTCEQAGNLLNHIE
LHSGEKPFKCHLCNYACRRRDAL

iTAG2v2, where one more residue in the linker between ZF1 and ZF2 of Ikaros was mutated:

iTAG2v2
(SEQ ID NO: 29)
PNVLMVHKRSHTGEIPLQCEICGFTCEQAGNLLNHIEL
HSGEKPFKCHLCNYACRRRDAL

iTAG2, where several residues present in the construct were mutated:

ITAG2
(SEQ ID NO: 43)
PNVLMVHNESHTGEIPLQCEICGFTCEQAGNLLNHIEL
HSGEIPFKCHLCNYACRRRDAL

Sequences were commercially synthesised (GeneArt, ThermoFischer Scientific) and inserted in a pLVX-TetOne-Puro lentiviral vector.

### Evaluation of EGFP-iTAG(1/2) Constructs

### Viral Transduction and Cell Line Generation

For viral particle production, HEK293T cells were plated 24 h prior to transfection and transfected with the pLVX-TetOne-Puro lentiviral vector, psPAX2 (packaging plasmid), pMD.2G using Lipofectamine™ 3000 (ThermoFisher). Virus-containing supernatants were harvested 48 hrs post-transfection and filtered through a 0.45 μm filter (Sartorius Stedim) to be stored in cryovials (ThermoFischer Scientific) at −80° C.

For cell transduction, HMEC cells were infected at MOI of 3 (Viral particle titre was determined using the Lenti-XTM GoStix™ kit (Clontech)) and polybrene (4 μg/ml) was used to increase the efficiency of transduction. Cells were cultured for at least two passages before inducing construct expression with 1 μg/ml of doxycycline (DOX) for 24 h. Fluorescence-activated cell sorting (FACS) was used to select for the top 30% of the cell population with the highest EGFP expression.

### Testing Degradation Using Flow Cytometry

Construct expression was induced by treating cells with 1 μg/ml DOX for 24 h. The cells were treated with IMiDs/CELMoDs at concentrations and time-points indicated for each experimental setting. Cells were collected, on ice, in sterile 5 ml polystyrene round-bottom tubes. DAPI (4′,6-diamidino-2-phenylindole; ThermoFisher Scientific) was added to the cell suspension at a ratio of 1:1000, as a marker for cell viability. The EGFP signal was measured on the BD LSR II flow cytometer (BD Biosciences). Further processing of the results was performed using FlowJo 10 Software (FlowJo).

### Testing Nuclear Localisation Using Immunofluorescence

HMEC cells were seeded in glass bottom 96-well plates (Perkin Elmer) at a 1000 cells/well and treated with 1 μg/ml DOX for 24 h to induce construct. Cells were fixed using 4% paraformaldehyde in PBS solution for 15 min at 37° C. and permeabilised with 0.05% Titon-X-100 detergent for 10 min at room temperature. Prior to proceeding to cell imaging, the cells were incubated for a min with 1 μg/ml DAPI (ThermoFisher Scientific) to stain the cell nuclei. Cells were visualised on the Zeiss Axio Vert.A1 FL-LED inverted epifluorescence microscope (Zeiss) and images were captured using the ZEN Blue software (Zeiss).

### Evaluation of CAR-iTAG(1/2) Constructs

### Constructs Generation and Cloning

Degrader tags iTAG1(DCD23) and iTAG2 were synthesised as gene blocks (IDT) and cloned into gammaretroviral vector SFG derived from MMLV using standard restriction site ligation. The vectors drive transcription of a single transcript in which the RQR8 epitope and CAR construct (anti-B7H3 scFv (TE9), CD8 transmembrane domain, CD28 co-stimulatory and CD3 signalling domain) and separate by a T2A ribosome skip site. Degrader tags were inserted directly following the CD3 signalling domain with no spacer sequences.

### Virus Production

Phoenix ampho packaging cells were grown and transfected with viral constructs containing CAR+/−tags using GeneJuice Transfection Reagent when around 80% confluent using standard protocols. Viral supernatant was harvested at 48 and 72 hours post transfection and pooled before snap freezing.

### Transduction of Primary Cells and Cell Lines:

Jurkat and 293T cell lines were grown in DMEM with 10% fetal calf serum and transduced with constructs containing CAR+/−degrader tag. Transduction efficiency was analysed 72 hours post transduction by flow cytometry, staining for the CD34 epitope encoded by the RQR8 gene.

PBMC-derived T cells from 2 healthy donor blood cones were cultured following CD56 depletion (day0) which was performed using Miltenyi CD56 depletion beads and magnetic negative selection using LD columns according to manufacturer's instructions. Post depletion, PBMC were stimulated with CD3+CD28 antibody beads (day 1) and IL-2 activation at 100iU/ml (day3). Cells were transduced on day 4 with CAR+/−degrader tag viruses using undiluted viral supernatant for PBMC and dilutions to yield multiplicity of infection ranging 1-5 for cell lines. Transduction efficiency was assessed 4 days post transduction by flow cytometry staining for RQR8 and CAR expression.

### IMiD Induced Degradation of CAR and CAR Functionality Experiments:

Degradation test in transduced Jurkat cell lines was conducted with addition of pomalidomide, iberdomide or lenalidomide at 10, 1, 0.1, 0.01 μM concentration or DMSO for 2, 4 or 24 hours, with CAR degradation assessed by flow cytometry. CAR expression in PBMC-derived T cells following degradation with 10, 1, 0.1, 0.01, 0.001 or 0 uM iberdomide after 24 hours, was assessed by flow cytometry. To assess CAR functionality+/−degrader tag, transduced T cells were cultured 24 hours with SUPT1+/−B7H3 at a 1:1 ratio. Supernatant was harvested, and IL-2 and IFNγ levels determined by ELISA.

### Flow Cytometric Analysis:

CAR surface expression for transduction efficiency and degradation experiments in primary cells and cell lines was quantified using his-tagged B7H3 protein in combination with anti his-tagged fluorescent antibody (J095G45, BioLegend), and directly fluorescently labelled anti CD34 antibody (QBEnd10, R&D), using LSR2 cytometer. Analysis used Flo-Jo software.

### Aiolos Peptide-Based TR-FRET Assay:

The production and purification methods for the proteins used in the TR-FRET assay were previously described in our publication on the discovery of the iTAG (Bouguenina, Nicolaou and Le Bihan et al., 2023. iTAG an optimized IMiD-induced degron for targeted protein degradation in human and murine cells. iScience, Volume 26, Issue 7. https://doi.org/10.1016/j.isci.2023.107059)¹⁹.

All TR-FRET assays were ran in Black 384-well ProxiPlate Plus (Perkin-Elmer, USA), in a buffer containing 20 mM HEPES pH 8.0, 150 mM NaCl, 0.5 mM TCEP, 0.05% Tween 20 and 0.05% BSA, with a final assay volume of 10 μL. An Echo E550 (Beckman Coulter, USA) acoustic liquid dispenser was used to create final concentration ranges from 82 μM down to 2.05 nM for iTAG1 and 27 μM down to 0.68 nM for iTAG2. Final concentrations of 5 nM WT full length CRBN/DDB1 complex, 750 nM Sulfo-Cy5 fluorescent Aiolos-based peptidic probe (Cambridge Research Biochemicals, UK), 750 nM Zn(OAc)² and 0.5 nM MAb Anti-6HIS-Terbium cryptate Gold (Cisbio, France) were added with a Tempest liquid handler (Formulatrix, USA). Finally, IMiDs were added to a final concentration of 10 μM with the Echo. Plates were sealed, centrifuged at 200 g for 1 min and stored overnight at 4° C. The TR-FRET signals were read using a PHERAstar FSX plate reader (BMG Labtech, Germany). Final signal was measured as the ratio:

$$TR - FRET signal = chanel 1 / chanel 2$$

Chanel1 at 665 nm representing a positive FRET and chanel2 at 620 nm representing the emission of the terbium when no FRET occurs.

### Size Exclusion Chromatography:

The production and purification methods for the proteins used to reconstitute the complex formed between CRBN/DDB1, iberdomide and iTAG2 by size exclusion chromatography (SEC) were previously described in our publication on the discovery of the iTAG (Bouguenina, Nicolaou and Le Bihan et al., 2023. iTAG an optimized IMiD-induced degron for targeted protein degradation in human and murine cells. iScience, Volume 26, Issue 7. https://doi.org/10.1016/j.isci.2023.107059)¹⁹.

SEC was performed using a Superose 6 5/150 GL column (Cytiva Life Sciences) mounted on an Agilent 1260 Infinity II LC System HPLC system. Two 60 μL samples were prepared by mixing purified CRBN/DDB1 and iTAG2 at 15 μM and 50 μM respectively in a buffer composed of 20 mM HEPES pH 7.0, 200 mM NaCl and 1 mM TCEP. 100 μM iberdomide was included in one of the samples and both were incubated on ice for 1 hour, then centrifuged at 21000 g for 10 minutes prior to injection to remove potential aggregates. The Superose 6 column was pre-equilibrated in the same buffer (20 mM HEPES pH 7.0, 200 mM NaCl and 1 mM TCEP) at a flow rate of 0.25 mL/min before injection of 50 μL of each sample. 100 μL fractions were collected and samples spanning the elution peaks were analysed by SDS-PAGE on precast NuPAGE 4-12% Bis-Tris gels (Invitrogen) using standard protocols, with SeeBlue Plus2 pre-stained protein standards (Invitrogen) loaded on the first lane of each gel.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent, or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

### REFERENCES

1. Bonger, K. M., Chen, L. C., Liu, C. W. & Wandless, T. J. Small-molecule displacement of a cryptic degron causes conditional protein degradation. Nat. Chem. Biol. 7, 531-537 (2011).
2. Chung, H. K. et al. Tunable and reversible drug control of protein production via a self-excising degron. Nat Chem Biol 11, 713-720 (2015).
3. Buckley, D. L. et al. HaloPROTACS: Use of Small Molecule PROTACs to Induce Degradation of HaloTag Fusion Proteins. ACS Chem Biol 10, 1831-1837 (2015).
4. Nabet, B. et al. Rapid and direct control of target protein levels with VHL-recruiting dTAG molecules. Nat. Commun. 11, 1-8 (2020).
5. Nabet, B. et al. The dTAG system for immediate and target-specific protein degradation. Nat. Chem. Biol. 14, 431-441 (2018).
6. Ege, N., Bouguenina, H., Tatari, M. & Chopra, R. Phenotypic screening with target identification and validation in the discovery and development of E3 ligase modulators. Cell Chem. Biol. 28, 283-299 (2021).
7. An, J. et al. PSILAC mass spectrometry reveals ZFP91 as IMiD-dependent substrate of the CRL4 CRBN ubiquitin ligase. Nat. Commun. 8, 1-11 (2017).
8. Sievers, Q. L. et al. Defining the human C2H2 zinc finger degrome targeted by thalidomide analogs through CRBN. Science 362, (2018).
9. Weber, E. W. et al. Transient rest restores functionality in exhausted CAR-T cells through epigenetic remodeling. Science 372, (2021).
10. Jan, M. et al. Reversible ON- and OFF-switch chimeric antigen receptors controlled by lenalidomide. Sci. Transl. Med. 13, (2021).
11. Ito, T. et al. Identification of a primary target of thalidomide teratogenicity. Science 327, 1345-1350 (2010).
12. Lu, G. et al. The myeloma drug lenalidomide promotes the cereblon-dependent destruction of ikaros proteins. Science 343, 305-309 (2014).
13. Krönke, J. et al. Lenalidomide causes selective degradation of IKZF1 and IKZF3 in multiple myeloma cells. Science 343, 301-305 (2014).
14. Krönke, J. et al. Lenalidomide induces ubiquitination and degradation of CK1alpha in del(5q) MDS. Nature 523, 183-188 (2015).
15. Gandhi, A. K. et al. Immunomodulatory agents lenalidomide and pomalidomide co-stimulate T cells by inducing degradation of T cell repressors Ikaros and Aiolos via modulation of the E3 ubiquitin ligase complex CRL4CRBN. Br. J. Haematol. 164, 811-821 (2014).
16. Matyskiela, M. E. et al. A novel cereblon modulator recruits GSPT1 to the CRL4 CRBN ubiquitin ligase. Nature 535, 252-257 (2016).
17. Krönke, J. et al. Lenalidomide causes selective degradation of IKZF1 and IKZF3 in multiple myeloma cells. Science 343, 301-305 (2014).
18. Krönke, J. et al. Lenalidomide induces ubiquitination and degradation of CK1α in del(5q) MDS. Nature 523, 183-188 (2015).
19. Bouguenina, Nicolaou and Le Bihan et al. iTAG an optimized IMiD-induced degron for targeted protein degradation in human and murine cells. iScience, Volume 26, Issue 7 (2023).

1. A degron tag comprising an amino acid sequence of LQCEICGFTCX₁QX₂GNLLX₃HIX₄LH (SEQ ID NO:66) wherein X₁, X₂, X₃ and, X₄ are not K, R or H.

2. The degron tag of claim 1, comprising an amino acid sequence of LQCEICGFTCX₁QX₂GNLLX₃HIX₄LH (SEQ ID NO:2) wherein:
X₁ is E or a conservative amino acid substitution thereof;
X₂ is A or a conservative amino acid substitution thereof;
X₃ is N or a conservative amino acid substitution thereof; and
X₄ is E or a conservative amino acid substitution thereof.

3. The degron tag of claim 2, wherein the tag comprises the amino acid sequence of
(SEQ ID NO: 5)
LQCEICGFTCEQAGNLLNHIELH.

4. The degron tag of claim 3, wherein the tag comprises the amino acid sequence of
(SEQ ID NO: 12)
LQCEICGFTCEQAGNLLNHIELHSG
or
(SEQ ID NO: 13)
LQCEICGFTCEQAGNLLNHIELHTG.

5. The degron tag of any preceding claim, wherein the tag comprises an additional N-terminal zinc finger alpha-helix subdomain and an additional C-terminal zinc finger beta-hairpin subdomain flanking the amino acid sequence of any one of SEQ ID NOs: 66, 2, 5, 12, or 13.

6. The degron tag of claim 5, wherein the additional C-terminal zinc finger beta-hairpin subdomain comprises the amino acid sequence CHLCNYACR (SEQ ID NO: 14) CHLCNYACQ (SEQ ID NO: 15), CHLCNYACRRRDAL (SEQ ID NO: 69) or CHLCNYACQRRDAL (SEQ ID NO: 70).

7. The degron tag of claim 5 or 6, wherein the additional N-terminal zinc finger alpha-helix subdomain comprises the amino acid sequence PNVLMVHX₅X₆SH (SEQ ID NO: 71) or FNVLMVHX₅X₆SH (SEQ ID NO:72); wherein X₅ and X₆ are not R, K or H.

8. The degron tag of claim 7, wherein the additional N-terminal zinc finger alpha-helix subdomain comprises the amino acid sequence PNVLMVHX₅X₆SH (SEQ ID NO: 16) or FNVLMVHX₅X₆SH (SEQ ID NO:17); wherein
X₅ is N or a conservative amino acid substitution thereof; and
X₆ is E or a conservative amino acid substitution thereof.

9. The degron tag of claim 8, wherein the additional N-terminal zinc finger alpha-helix subdomain comprises the amino acid sequence PNVLMVHNESH (SEQ ID NO: 22) or FNVLMVHNESH (SEQ ID NO: 23).

10. The degron tag of claim 8 or 9, wherein the tag comprises the amino acid sequence:
(SEQ ID NO: 73)
PNVLMVHX₅X₆SHTGEX₇PLQCEICGFTCX₁QX₂GNLLX₃HIX₄
LHSGEX₈PFKCHLCNYACRRRDAL,
or
(SEQ ID NO: 74)
FNVLMVHX₅X₆SHTGEX₇PLQCEICGFTCX₁QX₂GNLLX₃HIX₄
LHTGEX₈PFKCHLCNYACQRRDAL;
wherein X₇, and X₈ are not R, K or H.

11. The degron tag of claim 10, wherein the tag comprises the amino acid sequence:
PNVLMVHX₅X₆SHTGEX₇PLQCEICGFTCX₁QX₂GNLLX₃HIX₄LHSGEX₈PFKCHLCNYACRRR DAL (SEQ ID NO: 24), or
FNVLMVHX₅X₆SHTGEX₇PLQCEICGFTCX₁QX₂GNLLX₃HIX₄LHTGEX₈PFKCHLCNYACQRR DAL (SEQ ID NO: 45); wherein:
X₇ is I or a conservative amino acid substitution thereof; and
X₈ is I or a conservative amino acid substitution thereof.

12. The degron tag of claim 11, wherein the tag comprises the amino acid sequence:
(SEQ ID NO: 43)
PNVLMVHNESHTGEIPLQCEICGFTCEQAGNLLNH
IELHSGEIPFKCHLCNYACRRRDAL,
or
(SEQ ID NO: 44)
FNVLMVHNESHTGEIPLQCEICGFTCEQAGNLLNH
IELHTGEIPFKCHLCNYACQRRDAL.

13. The degron tag of any one of the preceding claims, wherein the tag has a length of about 23 to about 70 amino acids.

14. The degron tag of claim 10, wherein the tag has a length of about 23 to about 60 amino acids.

15. A fusion protein comprising a protein of interest and at least one degron tag of any one of claims 1 to 14.

16. The fusion protein of claim 15, wherein the degron tag is located C-terminal to the protein of interest.

17. The fusion protein of claim 15 or 16, wherein the protein of interest is a chimeric antigen receptor (CAR), a T cell receptor (TCR), a T cell receptor (TCR) fusion construct (TRuC), a T cell antigen coupler (TAC), a chimeric autoantibody receptor (CAAR), or an antibody-coupled T cell receptor (ACTR).

18. The fusion protein of claim 17, wherein the CAR fusion protein comprises from N-terminus to C-terminus:
a) an extracellular ligand binding domain;
b) a transmembrane domain;
c) a cytoplasmic domain comprising at least one intracellular signaling domain; and
d) the at least one degron tag of any one of claims 1 to 14.

19. The fusion protein of claim 18, wherein the extracellular ligand binding domain comprises an antibody or antigen binding fragment that is an scFv that binds to B7H3, the transmembrane domain is a CD8 transmembrane domain and the intracellular signaling domain is a CD3 signaling domain, and the CAR fusion protein further comprises a CD28 co-stimulatory domain.

20. A non-naturally occurring nucleic acid sequence encoding a degron tag of any one of claims 1 to 14 or a fusion protein of any one of claims 15 to 19.

21. A vector comprising a nucleic acid sequence of claim 20.

22. The vector of claim 21, wherein the vector is a viral vector, optionally wherein the viral vector is selected from the group consisting of: a retroviral vector, an adenoviral vector, an adeno-associated viral vector, a herpes simplex viral vector, a vaccinia viral vector, a picornaviral vector, and an alphaviral vector.

23. A cell which expresses a nucleic acid sequence of claim 20, or a vector of claim 21 or 22.

24. The cell of claim 23, wherein the cell is an immune effector cell.

25. The cell of claim 23 or 24, wherein the cell is selected from the group consisting of: a T cell, B cell, plasma cell, NK cell, NKT cell, innate lymphoid cell, macrophage, dendritic cell, monocyte, neutrophil, basophil, eosinophil, mast cell, hematopoietic progenitor cell, hematopoietic stem cell, other adult stem cell such as neural, cornea, muscle, skin, small intestine, colon, bone, mesenchyme, embryonic stem cell and an induced pluripotent stem cell.

26. The cell of any one of claims 23 to 25, wherein the cell is a mammalian cell, optionally wherein the cell is a human cell.

27. A pharmaceutical composition comprising a degron tag, fusion protein, nucleotide sequence, vector, or cell of any preceding claim, and a pharmaceutically acceptable excipient, carrier, adjuvant and/or diluent.

28. A pharmaceutical composition of claim 27 for use as a medicament.

29. A pharmaceutical composition of claim 27 for use in immune cell therapy.

30. A method of degrading a protein of interest comprising:
contacting a cell in vitro or in vivo with an effective amount of an immunomodulatory drug (IMiD) or a cereblon modulator (CELMoD), wherein the cell expresses a nucleic acid encoding a fusion protein of any one of claims 15 to 19.

31. A method of degrading a protein of interest comprising:
administering an effective amount of an immunomodulatory drug (IMiD) or a cereblon modulator (CELMoD) to a subject, wherein the subject has previously been treated via gene therapy causing at least some endogenous cells to express a nucleic acid encoding a fusion protein of any one of claims 15 to 19.

32. The method of claim 30 or 31, wherein said IMiD or CELMoD is thalidomide, pomalidomide, lenalidomide, CC-122, CC-220 or CC-885.


SEQUENCE LISTING (78 sequences)

SEQ ID NO: 1 - protein, 23 aa, synthetic construct
  REGION 1..23: note = Minimal degron sequence
LQCEICGFTCRQKGNLLRHIKLH

SEQ ID NO: 2 - protein, 23 aa, synthetic construct
  REGION 1..23: note = Degron tag
  VARIANT 11: note = E or a conservative amino acid substitution thereof
  VARIANT 13: note = A or a conservative amino acid substitution thereof
  VARIANT 18: note = N or a conservative amino acid substitution thereof
  VARIANT 21: note = E or a conservative amino acid substitution thereof
LQCEICGFTCXQXGNLLXHIXLH

SEQ ID NO: 3 - protein, 23 aa, synthetic construct
  REGION 1..23: note = Degron tag
  VARIANT 11: note = E or D
  VARIANT 13: note = A, G, P, I, L or V
  VARIANT 18: note = N, Q, C, S, T or M
  VARIANT 21: note = E or D
LQCEICGFTCXQXGNLLXHIXLH

SEQ ID NO: 4 - protein, 23 aa, synthetic construct
  REGION 1..23: note = Degron tag
  VARIANT 11: note = E or D
  VARIANT 13: note = A, G, or P
  VARIANT 18: note = N or Q
  VARIANT 21: note = E or D
LQCEICGFTCXQXGNLLXHIXLH

SEQ ID NO: 5 - protein, 23 aa, synthetic construct
  REGION 1..23: note = Sequence corresponding to the core region of the mutated ZFP91 ZF2 beta-hairpin subdomain and IKAROS ZF2 alpha-helix subdomain sequences of iTAG2, iTAGv1, and iTAGv2 degron tags
LQCEICGFTCEQAGNLLNHIELH

SEQ ID NO: 6 - protein, 25 aa, synthetic construct
  REGION 1..25: note = Degron tag
  VARIANT 11: note = E or a conservative amino acid substitution thereof
  VARIANT 13: note = A or a conservative amino acid substitution thereof
  VARIANT 18: note = N or a conservative amino acid substitution thereof
  VARIANT 21: note = E or a conservative amino acid substitution thereof
LQCEICGFTCXQXGNLLXHIXLHSG

SEQ ID NO: 7 - protein, 25 aa, synthetic construct
  REGION 1..25: note = Degron tag
  VARIANT 11: note = E or a conservative amino acid substitution thereof
  VARIANT 13: note = A or a conservative amino acid substitution thereof
  VARIANT 18: note = N or a conservative amino acid substitution thereof
  VARIANT 21: note = E or a conservative amino acid substitution thereof
LQCEICGFTCXQXGNLLXHIXLHTG

SEQ ID NO: 8 - protein, 25 aa, synthetic construct
  REGION 1..25: note = Degron tag
  VARIANT 11: note = E or D
  VARIANT 13: note = A, G, P, I, L or V
  VARIANT 18: note = N, Q, C, S, T or M
  VARIANT 21: note = E or D
LQCEICGFTCXQXGNLLXHIXLHSG

SEQ ID NO: 9 - protein, 25 aa, synthetic construct
  REGION 1..25: note = Degron tag
  VARIANT 11: note = E or D
  VARIANT 13: note = A, G, P, I, L or V
  VARIANT 18: note = N, Q, C, S, T or M
  VARIANT 21: note = E or D
LQCEICGFTCXQXGNLLXHIXLHTG

SEQ ID NO: 10 - protein, 25 aa, synthetic construct
  REGION 1..25: note = Degron tag
  VARIANT 11: note = E or D
  VARIANT 13: note = A, G, or P
  VARIANT 18: note = N or Q
  VARIANT 21: note = E or D
LQCEICGFTCXQXGNLLXHIXLHSG

SEQ ID NO: 11 - protein, 25 aa, synthetic construct
  REGION 1..25: note = Degron tag
  VARIANT 11: note = E or D
  VARIANT 13: note = A, G, or P
  VARIANT 18: note = N or Q
  VARIANT 21: note = E or D
LQCEICGFTCXQXGNLLXHIXLHTG

SEQ ID NO: 12 - protein, 25 aa, synthetic construct
  REGION 1..25: note = Sequence corresponding to the mutated ZFP91 ZF2 beta-hairpin subdomain and IKAROS ZF2 alpha-helix subdomain sequences of degron iTAG2, iTAGv1, and iTAGv2
LQCEICGFTCEQAGNLLNHIELHSG

SEQ ID NO: 13 - protein, 25 aa, synthetic construct
  REGION 1..25: note = Sequence corresponding to the mutated variant of the ZFP91 ZF2 beta-hairpin subdomain and AIOLOS ZF2 alpha-helix subdomain sequences of SuperDegron
LQCEICGFTCEQAGNLLNHIELHTG

SEQ ID NO: 14 - protein, 9 aa, synthetic construct
  REGION 1..9: note = Additional C-terminal zinc finger beta-hairpin subdomain
CHLCNYACR

SEQ ID NO: 15 - protein, 9 aa, synthetic construct
  REGION 1..9: note = Additional C-terminal zinc finger beta-hairpin subdomain
CHLCNYACQ

SEQ ID NO: 16 - protein, 11 aa, synthetic construct
  REGION 1..11: note = Additional N-terminal zinc finger alpha-helix subdomain
  VARIANT 8: note = N or a conservative amino acid substitution thereof
  VARIANT 9: note = E or a conservative amino acid substitution thereof
PNVLMVHXXSH

SEQ ID NO: 17 - protein, 11 aa, synthetic construct
  REGION 1..11: note = Additional N-terminal zinc finger alpha-helix subdomain
  VARIANT 8: note = N or a conservative amino acid substitution thereof
  VARIANT 9: note = E or a conservative amino acid substitution thereof
FNVLMVHXXSH

SEQ ID NO: 18 - protein, 11 aa, synthetic construct
  REGION 1..11: note = Additional N-terminal zinc finger alpha-helix subdomain
  VARIANT 8: note = N, Q, C, S, T or M
  VARIANT 9: note = E or D
PNVLMVHXXSH

SEQ ID NO: 19 - protein, 11 aa, synthetic construct
  REGION 1..11: note = Additional N-terminal zinc finger alpha-helix subdomain
  VARIANT 8: note = N, Q, C, S, T or M
  VARIANT 9: note = E or D
FNVLMVHXXSH

SEQ ID NO: 20 - protein, 11 aa, synthetic construct
  REGION 1..11: note = Additional N-terminal zinc finger alpha-helix subdomain
  VARIANT 8: note = N or Q
  VARIANT 9: note = E or D
PNVLMVHXXSH

SEQ ID NO: 21 - protein, 11 aa, synthetic construct
  REGION 1..11: note = Additional N-terminal zinc finger alpha-helix subdomain
  VARIANT 8: note = N or Q
  VARIANT 9: note = E or D
FNVLMVHXXSH

SEQ ID NO: 22 - protein, 11 aa, synthetic construct
  REGION 1..11: note = Additional N-terminal zinc finger alpha-helix subdomain
PNVLMVHNESH

SEQ ID NO: 23 - protein, 11 aa, synthetic construct
  REGION 1..11: note = Additional N-terminal zinc finger alpha-helix subdomain
FNVLMVHNESH

SEQ ID NO: 24 - protein, 60 aa, synthetic construct
  REGION 1..60: note = Degron tag
  VARIANT 8: note = N or a conservative amino acid substitution thereof
  VARIANT 9: note = E or a conservative amino acid substitution thereof
  VARIANT 15: note = I or a conservative amino acid substitution thereof
  VARIANT 27: note = E or a conservative amino acid substitution thereof
  VARIANT 29: note = A or a conservative amino acid substitution thereof
  VARIANT 34: note = N or a conservative amino acid substitution thereof
  VARIANT 37: note = E or a conservative amino acid substitution thereof
  VARIANT 43: note = I or a conservative amino acid substitution thereof
PNVLMVHXXSHTGEXPLQCEICGFTCXQXGNLLXHIXLHSGEXPFKCHLCNYACRRRDAL

SEQ ID NO: 25 - protein, 60 aa, synthetic construct; identical to SEQ ID NO: 24
  REGION 1..60: note = Degron tag
  VARIANT 27: note = E or D
  VARIANT 29: note = A, G, P, I, L or V
  VARIANT 34: note = N, Q, C, S, T or M
  VARIANT 37: note = E or D
  VARIANT 8: note = N, Q, C, S, T or M
  VARIANT 9: note = E or D
  VARIANT 15: note = I, L, V, G, A or P
  VARIANT 43: note = I, L, V, G, A or P

SEQ ID NO: 26 - protein, 60 aa, synthetic construct; identical to SEQ ID NO: 24
  REGION 1..60: note = Degron tag
  VARIANT 27: note = E or D
  VARIANT 29: note = A, G, or P
  VARIANT 34: note = N or Q
  VARIANT 37: note = E or D
  VARIANT 8: note = N or Q
  VARIANT 9: note = E or D
  VARIANT 15: note = I, L or V
  VARIANT 43: note = I, L or V

SEQ ID NO: 27 - protein, 60 aa, synthetic construct
  REGION 1..60: note = iTAG2v1
PNVLMVHKRSHTGERPLQCEICGFTCEQAGNLLNHIELHSGEKPFKCHLCNYACRRRDAL

SEQ ID NO: 28 - protein, 60 aa, synthetic construct
  REGION 1..60: note = Full length SuperDegron variant
FNVLMVHKRSHTGERPLQCEICGFTCEQAGNLLNHIELHTGEKPFKCHLCNYACQRRDAL

SEQ ID NO: 29 - protein, 60 aa, synthetic construct
  REGION 1..60: note = iTAG2v2
PNVLMVHKRSHTGEIPLQCEICGFTCEQAGNLLNHIELHSGEKPFKCHLCNYACRRRDAL

SEQ ID NO: 30 - protein, 60 aa, synthetic construct
  REGION 1..60: note = Full length SuperDegron variant
FNVLMVHKRSHTGEIPLQCEICGFTCEQAGNLLNHIELHTGEKPFKCHLCNYACQRRDAL

SEQ ID NO: 31 - protein, 60 aa, synthetic construct
  REGION 1..60: note = Full length iTAG1 variant
PNVLMVHKESHTGEIPLQCEICGFTCEQAGNLLNHIELHSGEKPFKCHLCNYACRRRDAL

SEQ ID NO: 32 - protein, 60 aa, synthetic construct
  REGION 1..60: note = Full length SuperDegron variant
FNVLMVHKESHTGEIPLQCEICGFTCEQAGNLLNHIELHTGEKPFKCHLCNYACQRRDAL

SEQ ID NO: 33 - protein, 60 aa, synthetic construct
  REGION 1..60: note = Full length iTAG1 variant
PNVLMVHNRSHTGEIPLQCEICGFTCEQAGNLLNHIELHSGEKPFKCHLCNYACRRRDAL

SEQ ID NO: 34 - protein, 60 aa, synthetic construct
  REGION 1..60: note = Full length SuperDegron variant
FNVLMVHNRSHTGEIPLQCEICGFTCEQAGNLLNHIELHTGEKPFKCHLCNYACQRRDAL

SEQ ID NO: 35 - protein, 60 aa, synthetic construct
  REGION 1..60: note = Full length iTAG1 variant
PNVLMVHKRSHTGEIPLQCEICGFTCEQAGNLLNHIELHSGEIPFKCHLCNYACRRRDAL

SEQ ID NO: 36 - protein, 60 aa, synthetic construct
  REGION 1..60: note = Full length SuperDegron variant
FNVLMVHKRSHTGEIPLQCEICGFTCEQAGNLLNHIELHTGEIPFKCHLCNYACQRRDAL

SEQ ID NO: 37 - protein, 60 aa, synthetic construct
  REGION 1..60: note = Full length iTAG1 variant
PNVLMVHNESHTGEIPLQCEICGFTCEQAGNLLNHIELHSGEKPFKCHLCNYACRRRDAL

SEQ ID NO: 38 - protein, 60 aa, synthetic construct
  REGION 1..60: note = Full length SuperDegron variant
FNVLMVHNESHTGEIPLQCEICGFTCEQAGNLLNHIELHTGEKPFKCHLCNYACQRRDAL

SEQ ID NO: 39 - protein, 60 aa, synthetic construct
  REGION 1..60: note = Full length iTAG1 variant
PNVLMVHKESHTGEIPLQCEICGFTCEQAGNLLNHIELHSGEIPFKCHLCNYACRRRDAL

SEQ ID NO: 40 - protein, 60 aa, synthetic construct
  REGION 1..60: note = Full length SuperDegron variant
FNVLMVHKESHTGEIPLQCEICGFTCEQAGNLLNHIELHTGEIPFKCHLCNYACQRRDAL

SEQ ID NO: 41 - protein, 60 aa, synthetic construct
  REGION 1..60: note = Full length iTAG1 variant
PNVLMVHNRSHTGEIPLQCEICGFTCEQAGNLLNHIELHSGEIPFKCHLCNYACRRRDAL

SEQ ID NO: 42 - protein, 60 aa, synthetic construct
  REGION 1..60: note = Full length SuperDegron variant
FNVLMVHNRSHTGEIPLQCEICGFTCEQAGNLLNHIELHTGEIPFKCHLCNYACQRRDAL

SEQ ID NO: 43 - protein, 60 aa, synthetic construct
  REGION 1..60: note = iTAG2
PNVLMVHNESHTGEIPLQCEICGFTCEQAGNLLNHIELHSGEIPFKCHLCNYACRRRDAL

SEQ ID NO: 44 - protein, 60 aa, synthetic construct
  REGION 1..60: note = Full length SuperDegron variant
FNVLMVHNESHTGEIPLQCEICGFTCEQAGNLLNHIELHTGEIPFKCHLCNYACQRRDAL

SEQ ID NO: 45 - protein, 60 aa, synthetic construct
  REGION 1..60: note = Degron tag
  VARIANT 27: note = E or a conservative amino acid substitution thereof
  VARIANT 29: note = A or a conservative amino acid substitution thereof
  VARIANT 34: note = N or a conservative amino acid substitution thereof
  VARIANT 37: note = E or a conservative amino acid substitution thereof
  VARIANT 8: note = N or a conservative amino acid substitution thereof
  VARIANT 9: note = E or a conservative amino acid substitution thereof
  VARIANT 15: note = I or a conservative amino acid substitution thereof
  VARIANT 43: note = I or a conservative amino acid substitution thereof
FNVLMVHXXSHTGEXPLQCEICGFTCXQXGNLLXHIXLHTGEXPFKCHLCNYACQRRDAL

SEQ ID NO: 46 - protein, 60 aa, synthetic construct; identical to SEQ ID NO: 45
  REGION 1..60: note = Degron tag
  VARIANT 27: note = E or D
  VARIANT 29: note = A, G, P, I, L or V
  VARIANT 34: note = N, Q, C, S, T or M
  VARIANT 37: note = E or D
  VARIANT 8: note = N, Q, C, S, T or M
  VARIANT 9: note = E or D
  VARIANT 15: note = I, L, V, G, A or P
  VARIANT 43: note = I, L, V, G, A or P

SEQ ID NO: 47 - protein, 60 aa, synthetic construct; identical to SEQ ID NO: 45
  REGION 1..60: note = Degron tag
  VARIANT 27: note = E or D
  VARIANT 29: note = A, G, or P
  VARIANT 34: note = N or Q
  VARIANT 37: note = E or D
  VARIANT 8: note = N or Q
  VARIANT 9: note = E or D
  VARIANT 15: note = I, L or V
  VARIANT 43: note = I, L or V

SEQ ID NO: 48 - protein, 23 aa, synthetic construct
  REGION 1..23: note = Degron tag
  VARIANT 11: note = R or substituted with E or a conservative amino acid substitution thereof
  VARIANT 13: note = K or substituted with A or a conservative amino acid substitution thereof
  VARIANT 18: note = R or substituted with N or a conservative amino acid substitution thereof
  VARIANT 21: note = K or substituted with E or a conservative amino acid substitution thereof
LQCEICGFTCXQXGNLLXHIXLH

SEQ ID NO: 49 - protein, 23 aa, synthetic construct
  REGION 1..23: note = Degron tag
  VARIANT 11: note = R or substituted with E or D
  VARIANT 13: note = K or substituted with A, G, P, I, L or V
  VARIANT 18: note = R or substituted with N, Q, C, S, T or M
  VARIANT 21: note = K or substituted with E or D
LQCEICGFTCXQXGNLLXHIXLH

SEQ ID NO: 50 - protein, 23 aa, synthetic construct
  REGION 1..23: note = Degron tag
  VARIANT 11: note = R or substituted with E or D
  VARIANT 13: note = K or substituted with A, G, or P
  VARIANT 18: note = R or substituted with N or Q
  VARIANT 21: note = K or substituted with E or D
LQCEICGFTCXQXGNLLXHIXLH

SEQ ID NO: 51 - protein, 23 aa, synthetic construct
  REGION 1..23: note = Degron tag
  VARIANT 11: note = R or substituted with E
  VARIANT 13: note = K or substituted with A
  VARIANT 18: note = R or substituted with N
  VARIANT 21: note = K or substituted with E
LQCEICGFTCXQXGNLLXHIXLH

SEQ ID NO: 52 - protein, 23 aa, synthetic construct
  REGION 1..23: note = Degron tag example
LQCEICGFTCEQKGNLLRHIKLH

SEQ ID NO: 53 - protein, 23 aa, synthetic construct
  REGION 1..23: note = Degron tag example
LQCEICGFTCRQAGNLLRHIKLH

SEQ ID NO: 54 - protein, 23 aa, synthetic construct
  REGION 1..23: note = Degron tag example
LQCEICGFTCRQKGNLLNHIKLH

SEQ ID NO: 55 - protein, 23 aa, synthetic construct
  REGION 1..23: note = Degron tag example
LQCEICGFTCRQKGNLLRHIELH

SEQ ID NO: 56 - protein, 23 aa, synthetic construct
  REGION 1..23: note = Degron tag example
LQCEICGFTCEQAGNLLRHIKLH

SEQ ID NO: 57 - protein, 23 aa, synthetic construct
  REGION 1..23: note = Degron tag example
LQCEICGFTCEQKGNLLNHIKLH

SEQ ID NO: 58 - protein, 23 aa, synthetic construct
  REGION 1..23: note = Degron tag example
LQCEICGFTCEQKGNLLNHIELH

SEQ ID NO: 59 - protein, 23 aa, synthetic construct
  REGION 1..23: note = Degron tag example
LQCEICGFTCRQAGNLLNHIKLH

SEQ ID NO: 60 - protein, 23 aa, synthetic construct
  REGION 1..23: note = Degron tag example
LQCEICGFTCRQAGNLLRHIELH

SEQ ID NO: 61 - protein, 23 aa, synthetic construct
  REGION 1..23: note = Degron tag example
LQCEICGFTCRQKGNLLNHIELH

SEQ ID NO: 62 - protein, 23 aa, synthetic construct
  REGION 1..23: note = Degron tag example
LQCEICGFTCEQAGNLLNHIKLH

SEQ ID NO: 63 - protein, 23 aa, synthetic construct
  REGION 1..23: note = Degron tag example
LQCEICGFTCEQAGNLLRHIELH

SEQ ID NO: 64 - protein, 23 aa, synthetic construct
  REGION 1..23: note = Degron tag example
LQCEICGFTCEQKGNLLNHIELH

SEQ ID NO: 65 - protein, 23 aa, synthetic construct
  REGION 1..23: note = Degron tag example
LQCEICGFTCRQAGNLLNHIELH

SEQ ID NO: 66 - protein, 23 aa, synthetic construct
  REGION 1..23: note = Degron tag
  VARIANT 11: note = not K, R or H
  VARIANT 13: note = not K, R or H
  VARIANT 18: note = not K, R or H
  VARIANT 21: note = not K, R or H
LQCEICGFTCXQXGNLLXHIXLH

SEQ ID NO: 67 - protein, 25 aa, synthetic construct
  REGION 1..25: note = Degron tag
  VARIANT 11: note = not R, K or H
  VARIANT 13: note = not R, K or H
  VARIANT 18: note = not R, K or H
  VARIANT 21: note = not R, K or H
LQCEICGFTCXQXGNLLXHIXLHSG

SEQ ID NO: 68 - protein, 25 aa, synthetic construct
  REGION 1..25: note = Degron tag
  VARIANT 11: note = not R, K or H
  VARIANT 13: note = not R, K or H
  VARIANT 18: note = not R, K or H
  VARIANT 21: note = not R, K or H
LQCEICGFTCXQXGNLLXHIXLHTG

SEQ ID NO: 69 - protein, 14 aa, synthetic construct
  REGION 1..14: note = Additional C-terminal zinc finger beta-hairpin subdomain
CHLCNYACRRRDAL

SEQ ID NO: 70 - protein, 14 aa, synthetic construct
  REGION 1..14: note = Additional C-terminal zinc finger beta-hairpin subdomain
CHLCNYACQRRDAL

SEQ ID NO: 71 - protein, 11 aa, synthetic construct
  REGION 1..11: note = Additional N-terminal zinc finger alpha-helix subdomain
  VARIANT 8..9: note = not R, K or H
PNVLMVHXXSH

SEQ ID NO: 72 - protein, 11 aa, synthetic construct
  REGION 1..11: note = Additional N-terminal zinc finger alpha-helix subdomain
  VARIANT 8..9: note = not R, K or H
FNVLMVHXXSH

SEQ ID NO: 73 - protein, 60 aa, synthetic construct; identical to SEQ ID NO: 24
  REGION 1..60: note = Degron tag
  VARIANT 27: note = not R, K or H
  VARIANT 29: note = not R, K or H
  VARIANT 34: note = not R, K or H
  VARIANT 37: note = not R, K or H
  VARIANT 8: note = not R, K or H
  VARIANT 9: note = not R, K or H
  VARIANT 15: note = not R, K or H
  VARIANT 43: note = not R, K or H

SEQ ID NO: 74 - protein, 60 aa, synthetic construct; identical to SEQ ID NO: 45
  REGION 1..60: note = Degron tag
  VARIANT 27: note = not R, K or H
  VARIANT 29: note = not R, K or H
  VARIANT 34: note = not R, K or H
  VARIANT 37: note = not R, K or H
  VARIANT 8: note = not R, K or H
  VARIANT 9: note = not R, K or H
  VARIANT 15: note = not R, K or H
  VARIANT 43: note = not R, K or H

SEQ ID NO: 75 - protein, 23 aa, synthetic construct
  REGION 1..23: note = Degron tag
  VARIANT 11: note = R or substituted with any amino acid other than R, K or H
  VARIANT 13: note = K or substituted with any amino acid other than R, K or H
  VARIANT 18: note = R or substituted with any amino acid other than R, K or H
  VARIANT 21: note = K or substituted with any amino acid other than R, K or H
LQCEICGFTCXQXGNLLXHIXLH

SEQ ID NO: 76 - protein, 60 aa, synthetic construct
  REGION 1..60: note = iTAG1
PNVLMVHKRSHTGERPLQCEICGFTCRQKGNLLRHIKLHSGEKPFKCHLCNYACRRRDAL

SEQ ID NO: 77 - protein, 60 aa, synthetic construct
  REGION 1..60: note = SuperDegron
FNVLMVHKRSHTGERPLQCEICGFTCRQKGNLLRHIKLHTGEKPFKCHLCNYACQRRDAL

SEQ ID NO: 78 - protein, 33 aa, synthetic construct
  REGION 1..33: note = Aiolos-based probe
ERPFQCNQCGASFTQKGNLLRHIKLHTGEKPFK